(19) United States
(12) Patent Application Publication
(10) Pub. No.: US 2024/0325596 A1
(43) Pub. Date: Oct. 3, 2024

(54) MINERAL-COATED SUBSTRATES FOR STABILIZATION OF RNA-BASED THERAPEUTIC COMPOSITIONS

(71) Applicant: Wisconsin Alumni Research Foundation, Madison, WI (US)

(72) Inventors: Joshua CHOE, Madison, WI (US); William MURPHY, Waunakee, WI (US)

(21) Appl. No.: 18/708,518

(22) PCT Filed: Nov. 9, 2022
(86) PCT No.: PCT/US2022/079534

§ 371 Date: May 8, 2024

Related U.S. Application Data
(60) Provisional application No. 63/277,421, filed on Nov. 9, 2021.

(51) Int. Cl. A61L 27/12; A61K 9/19; A61K 39/00; A61K 39/015; A61K 39/15; A61K 39/215; A61K 48/00; A61L 27/54; C12N 15/87

(52) U.S. Cl. CPC A61L 27/12; A61K 9/19; A61K 39/015; A61K 39/15; A61K 39/215; A61K 48/0025; A61L 27/54; C12N 15/87; A61K 2039/53; A61K 2039/575

(57) ABSTRACT

The present disclosure relates to ribonucleic acid (RNA)-based therapeutic compositions and in particular a messenger RNA (mRNA) based therapeutic composition. In one embodiment, the RNA-based therapeutic composition may include a mineral-coated substrate, RNA complexes bound to the mineral-coated substrate, and a lyoprotectant. The RNA complexes may include RNA complexed with a complexing agent. The composition may be lyophilized to a dry powder. The mineral-coated substrate may serve to stabilize and promote the transfection efficiency of the RNA complexes after lyophilization. In some embodiments, the mineral-coated substrate includes mineral-coated microparticles (MCMs). In other embodiments, the mineral-coated substrate is a mineral-coated glass vial.

### CROSS-REFERENCE TO RELATED APPLICATIONS

This application claims priority to U.S. Provisional Application No. 63/277,421 filed on Nov. 9, 2021, the contents of which is incorporated by reference in its entirety.

### GOVERNMENT SUPPORT

This invention was made with government support under NS109427 awarded by the National Institutes of Health. The government has certain rights in the invention.

### SEQUENCE LISTING

A Sequence Listing accompanies this application and is submitted as an XML file of the sequence listing named “960296.04350.xml” which is 9,098 bytes in size and was created on Nov. 8, 2022. The sequence listing is electronically submitted via Patent Center with the application and is incorporated herein by reference in its entirety.

### FIELD OF INVENTION

The present disclosure generally relates to ribonucleic acid (RNA)-based therapeutic compositions including mineral-coated substrates that stabilize and promote the stability and transfection efficiency of RNA complexes after lyophilization (freeze drying). The present disclosure further relates to methods of preparing such RNA-based therapeutic compositions, and to mineral-coated storage vessels containing such RNA-based therapeutic compositions.

### BACKGROUND

Messenger ribonucleic acid (mRNA)-based vaccines, such as the coronavirus disease 2019 (COVID-19) vaccines, offer viable and effective protection against infectious diseases and have potential for other therapeutic applications such as regenerative medicine. Relative to plasmid DNA (pDNA) gene delivery strategies, mRNA is safe and achieves high transfection efficiency in non-mitotic cells. One effective COVID-19 vaccine includes mRNA that encodes an antigen associated with severe acute respiratory syndrome coronavirus 2 (SARS-COV-2). Expression of the antigen in a treated subject trains the subject's immune system to recognize and neutralize the virus if an infection with SARS-COV-2 occurs. While effective, the application of mRNA vaccines may be restricted by the instability and inefficient in vivo delivery of mRNA. Specifically, RNA is inherently unstable and sensitive to hydrolysis, enzyme degradation, and changes in temperature. Ribonucleases which catalyze the degradation of RNA are ubiquitous in the environment and in vivo. In order to store and deliver active RNA-based therapeutics, extreme cold storage conditions (e.g., −20° C. to −90° C.) may be required from the time of manufacture up to the time of administration. The cold supply chain requirements for RNA-based therapeutics not only increases costs, but also leads to inequities around the globe as certain rural areas and developing countries may not have access to cold chain infrastructure.

Transfection agents have been developed to condense RNA and promote cellular uptake, avoiding cellular sensors for free nucleic acids thus improving delivery and nucleic acid translation. Some of the more effective complexing agents include compositions that are a mix of lipids (e.g. lipid nanoparticles) and polymers (e.g. lipopolyplexes). Addition of disaccharides (e.g., sucrose, trehalose) or other lyoprotectants may interact with the polar head groups of the lipopolyplex or lipid nanoparticles and improve long term storage stability after freeze-drying/lyophilization of the RNA complexes. However, even with the use of complexing agents and lyoprotectants, complications arising from aggregation and changes in particle sizes may reduce transfection efficiencies after freeze-drying the compositions.

Thus, there remains a need for improved strategies for stabilizing RNA-based therapeutic compositions at temperatures above current cold chain temperature conditions. There also remains a need for improved strategies for stabilizing RNA complexes after freeze drying/lyophilization. The present disclosure provides a technical solution to these needs.

### SUMMARY

Disclosed herein is a method of preparing a ribonucleic acid (RNA) and, in particular, a messenger ribonucleic acid (mRNA)-based therapeutic composition. The method may include incubating RNA with a complexing agent to form RNA complexes and incubating the RNA complexes with a mineral-coated substrate to bind the RNA complexes to the mineral-coated substrate. The method may further include suspending the bound RNA complexes in a solution containing a lyoprotectant to provide the RNA-based therapeutic composition and lyophilizing the RNA-based therapeutic composition to a dry powder.

Further disclosed herein is an RNA-based therapeutic composition. The RNA-based therapeutic composition may include a mineral-coated substrate and RNA complexes bound to the mineral-coated substrate, wherein the RNA complexes include RNA complexed with a complexing agent. The composition may further include a lyoprotectant. The RNA-based therapeutic composition may be lyophilized to a dry powder. In some embodiments, the dry powder of the composition may be stable at room temperature for an extended period of time without significant loss of a transfection efficiency of the RNA complexes.

Also disclosed herein is a mineral-coated storage vessel containing an RNA-based therapeutic composition for administration to a subject in need thereof. The mineral-coated storage vessel may include a glass vial having an abraded or sodium hydroxide treated inner surface, and a mineral coating layer applied to the inner surface of the glass vial. The RNA-based therapeutic composition contained within the glass vial may be in contact with the mineral coating layer. The RNA-based therapeutic composition may include a lyoprotectant and RNA complexed with a complexing agent. The RNA-based therapeutic composition may be lyophilized to a dry powder in the glass vial. In some embodiments, the dry powder of the composition may be stable at room temperature for an extended period of time without significant loss of a transfection efficiency of the RNA complexes.

### BRIEF DESCRIPTION OF THE DRAWINGS AND APPENDIX

FIG. 1 is a schematic representation of a messenger ribonucleic acid (mRNA)-based therapeutic composition including a mineral-coated substrate, in accordance with the present disclosure.

FIG. 2 is a schematic representation of an mRNA-based therapeutic composition with the mineral-coated substrate being a mineral-coated microparticle (MCM), in accordance with the present disclosure.

FIG. 3 is a schematic representation of an mRNA-based therapeutic composition with the mineral-coated substrate being a mineral-coated borosilicate glass vial, in accordance with the present disclosure.

FIG. 4 is a flowchart of steps that may be involved in preparing the mRNA-based therapeutic composition and administering the mRNA-based therapeutic composition to a subject, in accordance with the present disclosure.

FIG. 5 is a schematic representation of steps that may be involved in preparing mRNA complexes with MCMs and transfecting cells with the mRNA complexes and MCMs, in accordance with the present disclosure.

FIG. 6 is a schematic representation of preparing MCMs, in accordance with the present disclosure.

FIG. 7 is a flowchart of steps that may be involved in preparing a mineral-coated borosilicate glass vial, in accordance with the present disclosure.

FIG. 8 shows results demonstrating lyoprotection of mRNA complexes in the presence of MCMs, in accordance with the present disclosure. ‘Luminescence’ is a measure of the transfection efficiency of fresh (‘Fresh’) and lyophilized (‘Lyo’) plasmid DNA (‘pDNA’) and mRNA complexes (‘mRNA’) with (+MCM) or without (−) MCMs.

FIG. 9 shows results demonstrating promotion of lyoprotection of mRNA complexes with mineral coating layers on beta calcium triphosphate (β-TCP) microparticles, in accordance with the present disclosure. ‘Luminescence’ is a measure of the transfection efficiency of mRNA complexes with (uncoated) β-TCP microparticles (BTCP) or MCMs without dissacharides (−) or with varying concentrations of sucrose or trehalose dissacharides.

FIG. 10 shows changes in transfection efficiencies of mRNA therapeutic compositions with varying MCM:mRNA ratios and mRNA concentrations, in accordance with the present disclosure. ‘Luminescence’ is a measure of the transfection efficiency of therapeutic compositions having mRNA-lipid nanoparticle complexes with MCMs (MCM) or with MCMs prepared with 1 millimolar (mM) fluoride doped in the modified simulated body fluid (mSBF) (F-MCM) after a 24 hour transfection assay.

FIG. 11 shows changes in transfection efficiencies of mRNA therapeutic compositions with varying MCM:mRNA ratios and mRNA concentrations, in accordance with the present disclosure. ‘Luminescence’ is a measure of the transfection efficiency of therapeutic compositions having mRNA-lipopolyplex complexes with MCMs or with MCMs prepared with 1 mM fluoride doped in the mSBF (F-MCM) after a 24 hour transfection assay.

FIG. 12 shows results demonstrating the influence of complex binding time and complex formation time on binding between MCMs and mRNA-lipopolyplex (‘LPP’) and mRNA-lipid nanoparticle (‘LNP’) complexes, in accordance with the present disclosure.

FIG. 13 shows changes in mRNA complex sizes (mRNA-lipopolyplex complexes (left); and mRNA-lipid nanoparticle complexes (right)) with increasing complex formation time, in accordance with the present disclosure.

FIG. 14 shows changes in mRNA complex sizes (mRNA-lipopolyplex complexes (‘LPP’) complexes and mRNA-lipid nanoparticle complexes (‘LNP’)) with increasing complex formation time, in accordance with the present disclosure.

FIG. 15 shows binding of mRNA complexes (mRNA-lipopolyplex complexes (‘LPP Binding’) and mRNA-lipid nanoparticle complexes (‘LNP Binding’)) to MCMs in different binding conditions, in accordance with the present disclosure.

FIG. 16 shows results demonstrating an improvement in the lyoprotective effect of MCMs on mRNA complexes in the presence of different concentrations of disaccharide lyoprotectants, in accordance with the present disclosure. Frozen mRNA complexes without MCMs (‘Frozen Complex’), frozen mRNA complexes with MCMs (‘Frozen MCM’), and lyophilized mRNA complexes with MCMs (‘Lyo MCM’) with varying concentrations of sucrose (‘S’) or trehalose (‘T’) were used for transfection studies.

FIG. 17 shows the influence of calcium:phosphate ratio and carbonate molarity in the mSBF on the binding of mRNA-lipopolyplex complexes (‘LPP’) and mRNA-lipid nanoparticle complexes (‘LNP’) to MCMs, in accordance with the present disclosure.

FIG. 18 shows electron microscopy images of mineral coating layers formed with different concentrations of carbonate (4.2 mM and 25 mM) and different concentrations of calcium and phosphate in the mSBF, in accordance with the present disclosure. Bar scale=10 micrometers (μm).

FIG. 19 shows scanning electron microscopy images of MCMs produced with undoped mSBF having varying calcium:phosphate ratios (0.2×, 0.5×, 1×, 2.5×, and 5×), in accordance with the present disclosure.

FIG. 20 shows scanning electron microscopy images of MCMs produced with citrate-doped mSBF having varying calcium:phosphate ratios (0.2×, 0.5×, 1×, 2.5×, and 5×), in accordance with the present disclosure.

FIG. 21 shows scanning electron microscopy images of MCMs produced with fluoride-doped mSBF having varying calcium:phosphate ratios (0.5×, 1×, 2.5×, and 5×), in accordance with the present disclosure.

FIG. 22 shows scanning electron microscopy images of MCMs produced with citrate and fluoride doped mSBF having varying calcium phosphate ratios (0.2×, 0.5×, 1×, 2.5×, and 5×), in accordance with the present disclosure.

FIG. 23 shows scanning electron microscopy images of MCMs produced with silicate doped mSBF having varying calcium:phosphate ratios (0.2×, 0.5×, 1×, 2.5×, and 5×), in accordance with the present disclosure.

FIG. 24 shows scanning electron microscopy images of MCMs produced with citrate and silicate doped mSBF having varying calcium:phosphate ratios (0.2×, 0.5×, 1×, 2.5×, and 5×), in accordance with the present disclosure.

FIG. 25 shows pictures of mineral-coated glass vials prepared using sodium hydroxide pretreatment with (bottom) and without (top) surface abrasion with a Microblaster (50 μm silicon carbide particles), in accordance with the present disclosure.

FIG. 26 shows pictures of mineral-coated glass vials prepared using sodium metasilicate pretreatment (0-100 mM) with (bottom) and without (top) surface abrasion with a Microblaster (50 μm silicon carbide particles), in accordance with the present disclosure.

FIG. 27 shows bar graphs indicating the screening of disaccharides (1-50% w/v) for lyoprotection of mRNA loaded MCMs after freezing (F) or lyophilization (L) and storage for 7 days with or without 0.5×F-Cit MCMs using (A) Maltose (B) Sucrose (C) Trehalose (D) Comparison and (E) no disaccharides.

FIG. 28 shows luciferase activity 24 h post transfection. mRNA complexes in excipients 1-3 (E1-E3) were compared to 25% trehalose (25% T) for mRNA complexes (Com) and 0.5×-F-Cit MCMs stored lyophilized (Lyo) or frozen (Frz) at A.-B. −80° C. and 25° C. or I-P 4° C. after storage for 3 days to 6 months. Mean±standard deviation shown for bar graphs.

FIG. 29 (A)-(E) show the fold change in luciferase activity from baseline (3 days) after storage (3-150 days; −80°, 4° or 25° C.) of frozen (Frz) or lyophilized (Lyo) mRNA complexes bound to 0.5×F-Cit MCMs. Significance noted relative to d7. The graph in (F) shows luciferase activity 24 h post transfection of fresh mRNA complexes versus MCMs stored at −80° C. frozen (Frz) or lyophilized and stored at 25° C. for 6 months. Mean±standard deviation shown for bar graphs.

FIG. 30 shows luciferase activity (A) and metabolic activity (B) 24 h post transfection of lyophilized mRNA LNP and LPP complexes bound to regular and 0.5×-F-Cit MCMs stored at 25° C. for 28 days. Mean±standard deviation shown for bar graphs.

FIG. 31 (A)-(F) shows the transfection efficacy of LNP mRNA complexes after binding to MCMs of different compositions in PBS at a pH of 2-12 versus low serum media M. (G) shows the zeta potentials for mRNA LNP complexes alone or bound to regular vs. 0.5×F-Cit MCMs in PBS at a pH of 4 or 7.4. (H) shows the binding percentages of mRNA complexes bound to regular vs. 0.5×F-Cit MCMs in PBS at a pH of 4 or 7.4. Mean±standard deviation shown for bar graphs.

FIG. 32 shows calcium release (A) and phosphate release (B) in simulated body fluid from 0.5×MCMs with Cit, F, F-Cit compared to regular MCMs and β-TCP core material. (C)-(F) show luciferase activity normalized to metabolic activity of MSCs transfected with mRNA complexes or mRNA complexes+MCMs in the presence of increasing concentrations of inhibitors for (C) L-Type calcium channels (Nifedipine), (D) Phosphate uptake (Foscarnet), (E/F) Na/Citrate antiporter (PF-06761281). Mean±standard deviation shown for bar graphs. Dashed line represents baseline transfection efficacy without addition of drug.

FIG. 33 shows that MCMs stabilize mRNA complexes after lyophilization. (A) shows luciferase activity for fresh and lyophilized mRNA/pDNA complexes with or without MCMs. (B) shows transfection efficacy of lyophilized mRNA+/−complexing agent and +/−MCMs. Mean±standard deviation shown for bar graphs.

FIG. 34 shows Design of Experiments (DOE) for mRNA complex binding to MCMs optimizing the ratio of MCM to mRNA complexes (μg:μg) and mRNA complex concentration (μg/mL) to maximize transfection efficacy. (A), (E): Luminescence and (B), (F): Metabolic activity for LNP (top row) and LPP (bottom row) loaded MCMs and F-MCMs. (C)-(D), (G)-(H): Linear regression for MCMs/F-MCMs for LNPs and LPPs based on the predicted vs actual transfection efficacy as measured by luciferase activity. Mean±standard deviation shown for bar graphs.

FIG. 35 illustrates the strategy for creation of a library of mineral coatings varying calcium to phosphate rations (0.2×, 0.5×, 1×, 2.5×, and 5×) as well as the addition of fluoride (F), citrate (Cit) and silicate (Si) as dopants.

FIG. 36 (A) shows scanning electron micrographs (300 nm scale) of 40 mineral coated microparticles varying calcium:phosphate (0.2×, 0.5×, 1×, 2.5×, and 5×) and introduction of dopants (no dopant, citrate, fluoride, fluoride-citrate, silicate, silicate-citrate, silicate-fluoride, and silicate-fluoride-citrate). (B)-(D) shows representative structures of plate-like ((B). 2.5×MCM), needle-like ((C) 0.5×F-MCM) and hybrid ((D) 0.5×F-Cit-MCM) structures seen with electron microscopy. (E) shows luciferase activity after 24 hours of treatment with lyophilized mRNA complexes bound to a library of 40 different mineral coated microparticles and stored for 72 h compared to the uncoated core material (β-TCP), mRNA complexes (com) alone and untreated cells (−/−). (F) shows the metabolic activity of cells treated as in (E). (G) shows fluorescence micrographs of eGFP mRNA complexes loaded onto MCMs and delivered to hMSCs for 24 hours (100 μm scale). Mean±standard deviation shown for bar graphs.

FIG. 37 shows binding percentages as measured by fluorescence depletion of LNP and LPP complexes to regular MCMs (A) with binding times of 15-120 minutes (B) complex formation times of 5-20 minutes prior to 30 minutes of binding to regular MCMs. (C)-(K) show dynamic light scattering determination of mRNA complex size during 5-20 minutes of complex formation. Mean±standard deviation shown for bar and line graphs.

FIG. 38 (A) shows binding and elution of LPP mRNA complexes as measured by change in fluorescence measurements with binding to regular MCMs relative to unbound complexes for excipients 1 (E1) and 2 (E2) without disaccharides (−) and with sucrose(S) and trehalose (T). (B) shows binding and elution of LPP mRNA complexes in different solutions (25 mM each) based on hydroxyapatite chromatography. (C) shows binding of LPP mRNA complexes under different concentrations (25-100 mM) of CaCl₂) (C) and MES (M) or with 25 mM phosphate (P). (D) shows binding and elution of LNP mRNA complexes to regular MCMs. (E) shows transfection efficiency and (F) metabolic activity 24 hours after delivery of luciferase mRNA LNP complexes with 0.5×F-Cit MCMs.

FIG. 39 (A) shows Crystal Structure (X-ray Diffraction), (B)-(C) chemical composition ((B) Energy Dispersive Spectroscopy, (C) Fourier Transform Infrared Spectroscopy) of 0.5×-F-Cit MCMs, matched controls, regular MCMs and uncoated β-TCP core material.

FIG. 40 (A) shows that mineral coated glass vials could be used as an alternate storage strategy for lyophilized mRNA therapeutics allowing for easier bedside translation without injection of microparticles. (B)-(E) shows the formation of mineral coating on borosilicate glass vials that are either untreated (B-C) or abraded with 20 μm sand particles prior to mineralization with F-Cit 9.5×mSBF (D-E).

FIG. 41 shows elution of G. Luc mRNA LNPs bound to 0.5×F-Cit MCMs in the following elution conditions: 0.03-2 mM NaPO₄ in 2 M NaCl (Buffer A, pH 7), 7.5-500 mM NaPO₄ with 150 mM NaCl (Buffer B, pH 6.8), 7.5-400 mM NaPO₄ with 1 mM EDTA (Buffer C, pH 7.0), 7.5-500 mM NaPO₄ (Buffer D, pH 6.8) or 7.5-400 mM KH₂PO₄, 1 mM EDTA (Buffer E).

### DETAILED DESCRIPTION

As used herein, “cold chain” refers to an unbroken supply chain for a product controlled at a low temperature or low temperature range to maintain the quality of the product via an uninterrupted series of refrigerated production, storage, and distribution activities along with associated equipment and logistics.

“Lyoprotection”, as used herein, refers to the protection of a substance undergoing lyophilization against damage.

A “lyoprotectant”, as used herein, refers to a substance that that has lyoprotective properties.

As used herein, “mRNA” refers to messenger ribonucleic acid. However, other types of RNA may be utilized herein, such as guide RNA (gRNA), microRNA (miRNA), short hairpin (shRNA), RNA aptamers, small interfering RNA (siRNA) or other RNA based products or therapeutics.

As used herein, “mRNA complexes” or “RNA complexes” refer to complexes of RNA with a complexing agent such as a lipopolyplex or a lipid nanoparticle complexing agent. The complexes may substitute any other type of RNA, such as gRNA, miRNA, or siRNA for mRNA.

A “complexing agent”, as used herein, refers to a transfection agent that binds to mRNA or other type of RNA and promotes cell transfection efficiency. Complexing agents used herein include, but are not limited to, lipopolyplexes and lipid nanoparticles.

“Lipopolyplex”, as used herein, refers to a core-shell structure composed of nucleic acid, polycation, and lipid.

A “microparticle”, as used herein, refers to a particle that has a particle size in the micrometer range (or a particle size between about 0.2 μm and about 1000 μm).

As used herein, a “mineral-coated substrate” is a substrate coated with a mineral coating layer containing at least calcium, phosphate, and carbonate.

“Complex formation” as used herein refers to the complex formation between mRNA or other type of RNA and the complexing agent.

As used herein, “binding” refers to binding between mRNA complexes and a mineral-coated substrate (e.g., a mineral-coated microparticle or a mineral-coated glass vial).

As used herein, “mSBF” refers to modified simulated body fluid. The modified simulated body fluid is a solution with ion concentrations approximately equal to that of human blood plasma. As used herein, an mSBF differs from a standard simulated body fluid (SBF) in its concentrations of one or more of calcium, phosphate, carbonate, and dopants such as fluoride, silicates, and citrates.

As used herein, a “subject” refers to a live human or animal subject undergoing treatment.

The present disclosure addresses the storage and stability issues presented by RNA-based therapeutics including, but not limited to, mRNA-based vaccines. The present disclosure is directed to the use of mineral-coated substrates, including mineral-coated microparticles and mineral-coated glass vials, which stabilize and promote the transfection efficiency of therapeutic mRNA complexes after lyophilization. The mRNA complexes may be adsorbed to the mineral coating layer of the mineral-coated substrate, and the resulting freeze-dried composition may have greater stability at storage temperatures well above current cold chain storage temperatures. In some embodiments, the resulting freeze-dried compositions may be stored and handled at room temperature, thereby enabling easier handling, eliminating the need for cold chain storage and transport, and enhancing access to these products. The technology disclosed herein could be used to stabilize mRNA complexes of existing vaccines and those of future RNA-based therapeutics.

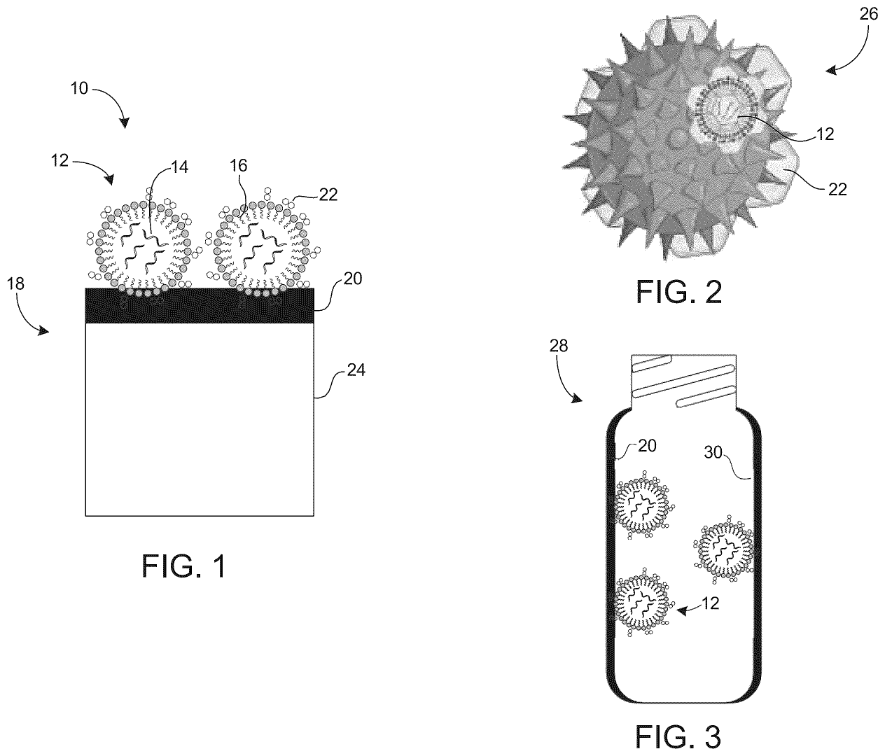

Referring to the drawings, and with specific reference to FIG. 1, a messenger ribonucleic acid (mRNA)-based therapeutic composition 10 is shown. In some embodiments, the mRNA-based therapeutic may be some other type of RNA-based therapeutic, such as gRNA, miRNA, siRNA or even an aptamer. In some embodiments, the mRNA-based therapeutic composition 10 may be a vaccine against a particular infectious disease such as, but not limited to, coronavirus 2019 (COVID-19), human immunodeficiency virus (HIV), malaria, or other known or emerging infectious disease. The mRNA-based therapeutic composition 10 may include mRNA complexes 12 formed of mRNA 14 complexed with a complexing agent 16, and a mineral-coated substrate 18 having one or more mineral coating layers 20 that stabilize the mRNA complexes 12 and improve transfection efficiency of the composition after freeze drying/lyophilization. Further included in the composition 10 may be one or more lyoprotectants 22 that protect the mRNA complexes 12 from damage that may occur during freeze drying/lyophilization. The composition 10 may be provided as a dry medication or powder that is reconstituted in solution prior to administration to a subject.

The mRNA 14 may include any therapeutically active mRNA. In some embodiments, the mRNA 14 may encode an antigen associated with severe acute respiratory syndrome coronavirus 2 (SARS-COV-2). In other embodiments, the mRNA 14 may encode an antigen associated with HIV, malaria, or another known or emerging infectious disease. Suitable mRNAs also include RNAs with chemical modifications including labels (e.g., fluorescent labels, protein labels, radioactive labels, metal labels, nanoparticle labels, etc.) and/or chemically modified bases such as, but not limited to, 5-methylcytidine, pseudouridine, 2-thiouridine, and Ni-methyl-pseudouridine. In other embodiments, the mRNA 14 may be substituted by gRNA, miRNA, shRNA, aptamer, siRNA or any other RNA-based therapeutic.

The complexing agent 16 may be a transfection reagent that forms a complex with the mRNA 14 and improves transfection efficiency. Suitable complexing agents may include positively charged lipid-based complexes such as lipopolyplexes (LPP) and lipid nanoparticles (LNP). Such an LNP may comprise DLin-MC3-DMA, DMG-PEG (2000), and 1,2-DSPC (LNP-MC3 Exploration Kit, Cayman Chemical), SM-102, DMG-PEG (2000), 1,2-DSPC (LNP-102 Exploration Kit, Cayman Chemical), or any other suitable lipids. The lyoprotectant 22 may be a disaccharide that interacts with the polar head groups of the complexing agent (LPP or LNP) to prevent damage during lyophilization and help with long term storage of the composition 10. Preferred lyoprotectants include trehalose or sucrose. In some embodiments, the therapeutic composition 10 may contain from about 5 millimolar (mM) to about 1 molar (M), or about 30 mM to about 1 M of the disaccharide (prior to lyophilization). In one specific embodiment, the composition 10 may contain about 150 mM trehalose. In another embodiment, the composition 10 may contain about 250 mM or about 254 mM sucrose. Other types of lyoprotectants may also be used including, but not limited to, glucose, maltose, lactose, inositol, dextran, hydroxypropyl-B-cyclodextrin, polyethylene glycol, and combinations thereof.

The mineral-coated substrate 18 may include a substrate 24 having the mineral coating layer 20 applied to one or more surfaces thereof. The mineral coating layer 20 may have different ratios of calcium, phosphate, and carbonate. The calcium:phosphate ratio in the mineral coating layer 20 may vary from about 0.1 to about 10, or from about 2 to about 5. The carbonate concentration of the mineral coating layer 20 may vary from about 1 mM to about 150 mM, or from about 3 mM to about 100 mM. Other ratios/concentrations of calcium, phosphate, and carbonate may be used in alternative embodiments. Different morphologies of the mineral coating layer 20 (e.g., plate-like, spherulite-like, etc.) may be achieved by varying the amounts and ratios of calcium, phosphate, and carbonate. For example, a high carbonate concentration may result in a mineral coating layer having a plate-like structure, whereas a low carbonate concentration may result in a mineral coating layer having a spherulite-like structure.

In some embodiments, the mineral-coated substrate 18 may include a mineral-coated microparticle (MCM) 26 (see FIG. 2). The MCM 26 may include a core material in the form of a microparticle coated with the mineral coating layer 20. Suitable core materials on which the mineral coating layer 20 is formed may include polymers, ceramics, metals, glass, and combinations thereof in the form of microparticles. Non-limiting examples of suitable microparticles include ceramics (e.g., hydroxyapatite, beta-tricalcium phosphate (β-TCP), magnetite, neodymium), plastics (e.g., polystyrene, poly-caprolactone), hydrogels (e.g., polyethylene glycol, poly(lactic-co-glycolic acid) and the like, and combinations thereof. Particularly suitable core materials include those that are dissolved in vivo such as β-TCP and hydroxyapatite. The mRNA complexes 12 may be adsorbed to the mineral coating layer 20 of the MCM 26. Upon cell transfection or administration, the mRNA complexes 12 adsorbed to the mineral coating layer 20 may be released as the mineral coating layer degrades. Upon introduction to the cytoplasm (via endocytosis, micropinocytosis or other mechanism), the mRNAs 14 may proceed to translation for protein production. After translation and processing, the MCMs 26 used for initial delivery of the mRNA complexes 12 may bind and sequester the secreted protein. The protein may be released from the MCM 26 over time back to the cell, prolonging the biological response.

In other embodiments, the mineral-coated substrate 18 may be a mineral-coated glass vial or storage vessel 28. The glass vial 28 may be a borosilicate glass vial or another type of glass vial. In this aspect, the mineral coating layer 20 may be applied to an inner surface 30 of the glass vial 28, and the mRNA complexes 12 may be adsorbed to the mineral coating layer 20. The composition 10 may be stored as a dry powder in the glass vial 28, with the mineral coating layer 20 serving to stabilize and promote the transfection efficiency of the mRNA complexes 12 after lyophilization. Reconstitution in solution at the time of administration to a subject may remove the mRNA complexes 12 from the mineral coating layer 20, such that only reconstituted mRNA complexes 12 are delivered to the subject. In other embodiments, at least some of the mineral coating layer 20 may be dissolved into the reconstitution solution and delivered to the subject. The reconstitution buffer may also be called the elution buffer and generally includes between 0.03-2 M NaPO₄ or KH₂PO₄, or alternatively between 0.1 mM and 1M NaPO₄ or KH₂PO₄ between 5 mM and 500 mM NaPO₄ or KH₂PO₄, 10 mM and 400 mM NaPO₄ or KH₂PO₄. The pH of the elution buffer may be a pH of 6.5, 6.7, 6.8. 6.9. 7.0. 7.1, 7.2, 7.3, 7.4, 7.5. The elution buffer may further comprise a salt such as between 100 mM and 2M NaCl and a calcium chelator such as EDTA at a concentration between 0.5 mM and 2.0 mM.

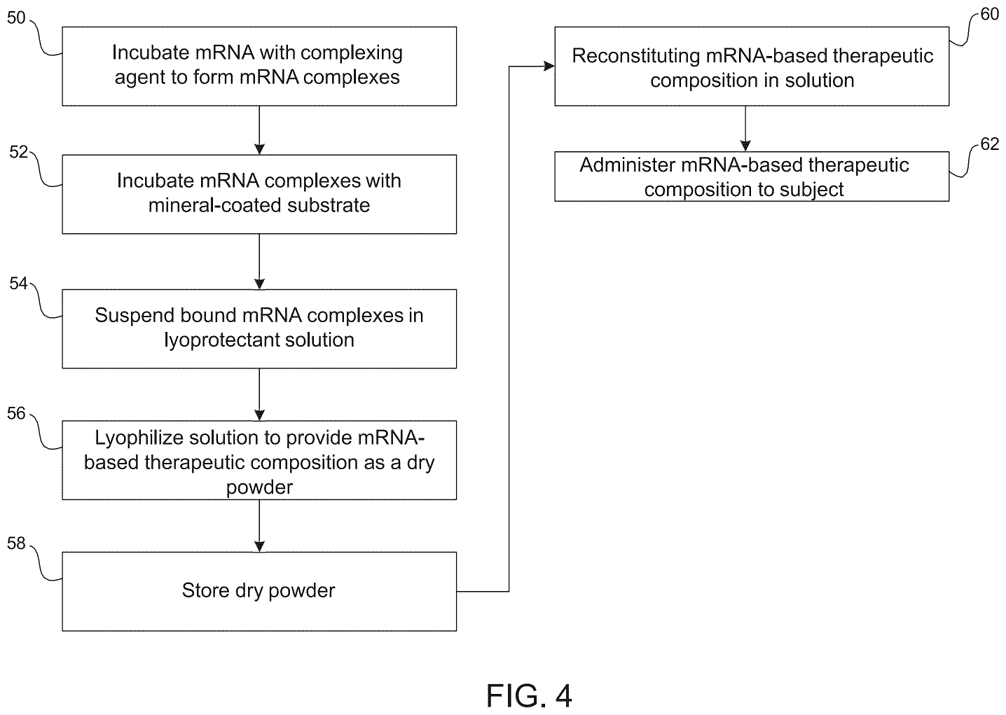

A method of preparing the mRNA-based therapeutic composition 10 and administering the composition 10 to a subject is shown in FIG. 4. At a block 50, the mRNA 14 may be incubated with the complexing agent 16 to form the mRNA complexes 12. The resulting mRNA complexes 12 may be subsequently incubated with the mineral-coated substrate 18 to bind the mRNA complexes 12 to the mineral-coated substrate 18 (block 52). At a next block 54, the bound mRNA complexes may be suspended in a solution containing the lyoprotectant 22. In some embodiments, the solution containing the lyoprotectant may contain about 30 mM to about 1 M trehalose as the lyoprotectant. In one specific embodiment, the bound mRNA complexes may be suspended in a solution of 150 mM trehalose. In other embodiments, the solution containing the lyoprotectant 22 may contain about 30 mM to about 1 M sucrose as the lyoprotectant. In one embodiment, the bound mRNA complexes may be suspended in a solution of about 250 mM or about 254 mM sucrose. Other lyoprotectants or lyoprotectant concentrations may also be used as described above. At a next block 56, the solution may be lyophilized to provide the mRNA-based therapeutic composition 10 as a dry powder.

The resulting dry powder may be stored according to a block 58. The use of the mineral-coated substrate 18 may stabilize the resulting dry powder after lyophilization and elevate the temperature stability for storage of the composition 10 over that of the mRNA complexes 12 alone. In some aspects, the resulting composition 10 may be stable at room temperature (or other temperatures above cold chain storage conditions) for extended periods of time, eliminating the need for cold chain transfer and storage conditions and facilitating distribution to wider populations. At the time of administration, the dry powder may be reconstituted in solution (block 60). In some embodiments, the solution used for reconstitution may be a salt solution, such as a saline solution or an ammonium salt solution. If the mineral-coated substrate 18 includes MCMs 26, then the block 60 may involve reconstituting the MCMs 26 and the mRNA complexes 12 in the solution. If the mineral-coated substrate 18 is the mineral-coated glass vial 28, then the block 60 may involve disrupting the binding interaction between the mineral coating layer 20 and the mRNA complexes 12 with the salt solution and dissolving the mRNA complexes 12 on their own (without the mineral coating) into the salt solution. After reconstituting the mRNA therapeutic composition 10, the solution may be administered to the subject (block 62) via injection (e.g., subcutaneous injection, intramuscular injection, intravenous etc.). Other types of administration such as, but not limited to, oral administration or intranasal administration may also be used in some aspects.

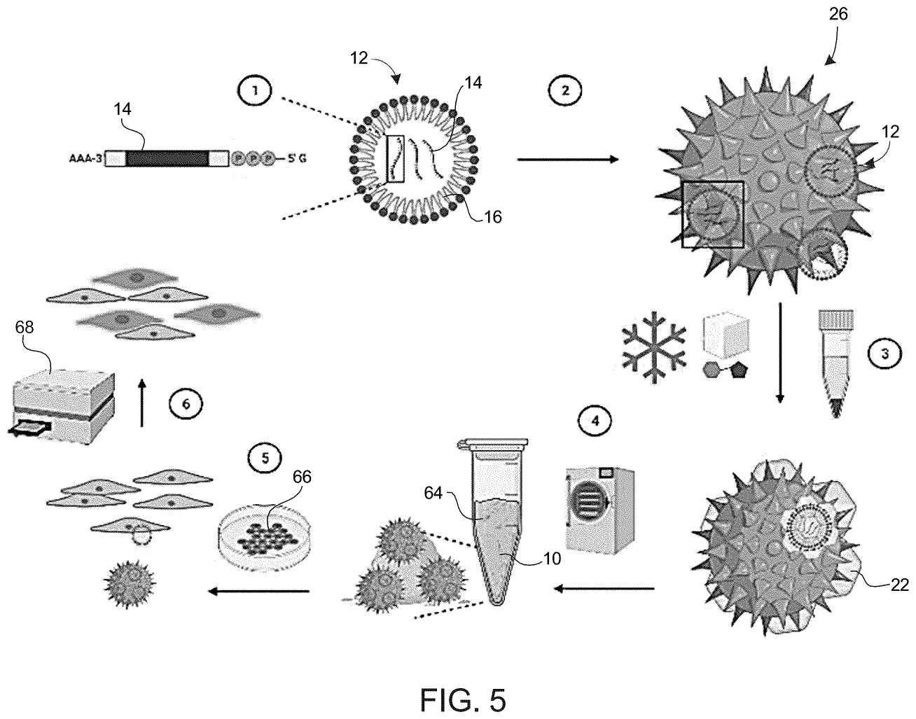

An exemplary method of preparing the mRNA complexes 12 with the MCMs 26 is shown in FIG. 5. At step 1, the mRNA 14 may be incubated with the complexing agent 16 to form the mRNA complexes 12. At step 2, the mRNA complexes 12 may be incubated with the MCMs 26 to bind the mRNA complexes 12 with the MCMs 26. The bound mRNA complexes 12 may then be suspended in the lyoprotectant solution and frozen (step 3). At step 4, the frozen solution may be lyophilized to provide the composition 10 as a dry powder 64. In vitro transfection of cells 66 may be carried out with the resulting composition 10 (steps 5 and 6). In vitro studies of transfection efficiency may be carried out if the mRNA 14 encodes a detectable protein, such as a luminescent protein, or provides another detectable output for translation. For instance, the mRNA 14 may encode a bioluminescent protein that can be quantified with a luminescence plate reader 68. In one specific embodiment for in vitro studies, the mRNA may encode Gaussia luciferase or another luminescent protein, such as Green Fluorescent Protein (GFP). If, however, the dry powder 64 of the mRNA complexes 12 and the MCMs 26 is used for transfection in a living subject, it may be reconstituted in solution and administered to the subject as described above. It will be understood that the order of the steps shown in FIGS. 4-5 are exemplary, and that some steps may be carried out in different orders or simultaneously in certain embodiments.

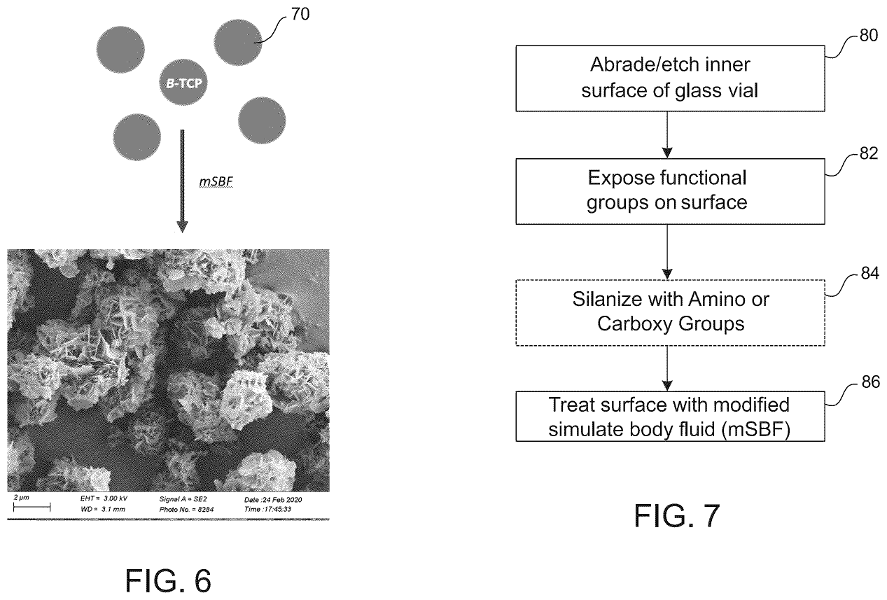

A method of preparing the MCMs is shown in FIG. 6. In this embodiment, microparticles used as the core material are β-TCP microparticles 70. The β-TCP microparticles 70 may have a particle size that ranges from about 1 micron to about 3 microns, although larger or smaller particle sizes may also be used in some arrangements. The β-TCP microparticles 70 may be suspended in modified simulated body fluid (mSBF) and incubated for a period of time to form the mineral coating layer 20 on the microparticles 70 (see Example 3). The mineral coating layer 20 may be formed on the microparticles 70 over a period of minutes to days (see Example 3).

The mSBF used for the preparation of the mineral coating layer 20 may include from about 5 mM to about 12.5 mM calcium ions, including from about 7 mM to about 10 mM calcium ions, and including about 8.75 mM calcium ions; from about 2 mM to about 12.5 mM phosphate ions, including from about 2.5 mM to about 7 mM phosphate ions, and including from about 3.5 mM to about 5 mM phosphate ions; and from about 4 mM to about 100 mM carbonate ions. In some embodiments, the mSBF may further include about 145 mM sodium ions, from about 6 mM to about 9 mM potassium ions, about 1.5 mM magnesium ions, from about 150 mM to about 175 mM chloride ions, about 4 mM bicarbonate (HCO₃⁻) ions, and about 0.5 mM sulfate (SO₄²⁻) ions. The pH of the mSBF may range from about 4 to about 7.5, including from about 5.3 to about 6.8, including from about 5.7 to about 6.2, and including from about 5.8 to about 6.1.

A suitable mSBF may include, for example, about 145 mM sodium ions, about 6 mM to about 9 mM potassium ions, about 5 mM to about 12.5 mM calcium ions, about 1.5 mM magnesium ions, about 150 mM to about 175 mM chloride ions, about 4.2 mM bicarbonate ions, about 2 mM to about 5 mM hydrogen phosphate (HPO₄²⁻) ions, and about 0.5 mM sulfate ions. The pH of the mSBF may be from about 5.3 to about 7.5, including from about 6 to about 6.8.

Another suitable mSBF may include, for example, about 145 mM sodium ions, about 6 mM to about 17 mM potassium ions, about 5 mM to about 12.5 mM calcium ions, about 1.5 mM magnesium ions, about 150 mM to about 175 mM chloride ions, about 4.2 mM to about 100 mM bicarbonate ions, about 2 mM to about 12.5 mM phosphate ions, and about 0.5 mM sulfate ions. The pH of the mSBF may be from about 5.3 to about 7.5, including from about 5.3 to about 6.8.

Another suitable mSBF may include, for example, about 145 mM sodium ions, about 6 mM to about 9 mM potassium ions, about 5 mM to about 12.5 mM calcium ions, about 1.5 mM magnesium ions, about 60 mM to about 175 mM chloride ions, about 4.2 to about 100 mM bicarbonate ions, about 2 mM to about 5 mM phosphate ions, and about 0.5 mM sulfate ions. The pH of the mSBF may be from about 5.8 to about 6.8, including from about 6.2 to about 6.8.

Yet another suitable mSBF may include about 145 mM sodium ions, about 9 mM potassium ions, about 12.5 mM calcium ions, about 1.5 mM magnesium ions, about 172 mM chloride ions, about 4.2 mM bicarbonate ions, about 5 mM to about 12.5 mM phosphate ions, and from about 4 mM to about 100 mM carbonate (CO₃²⁻) ions. The pH of the mSBF may be from about 5.3 to about 6.0.

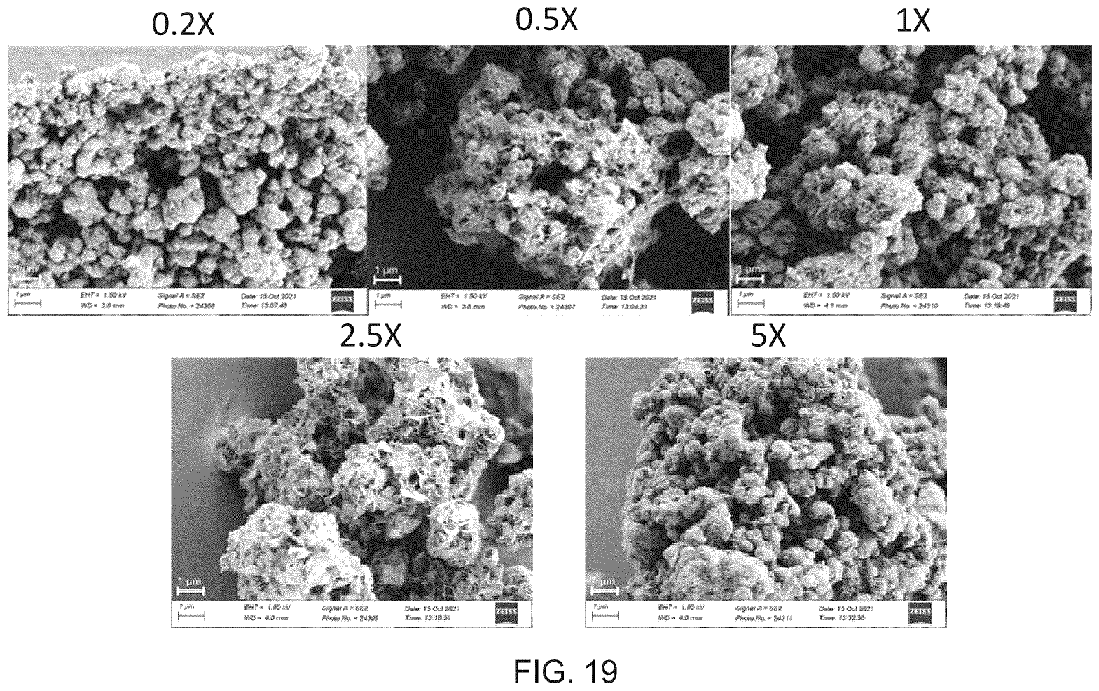

With reference to Table 1, the base composition for the mSBF (or the ‘regular’ mSBF) may include 141 mM sodium chloride (NaCl), 4 mM potassium chloride (KCl), 0.5 mM magnesium sulfate (MgSO₄), 1 mM magnesium chloride (MgCl₂), 4.2 mM sodium bicarbonate (NaHCO₃), 20 mM 2-[4-(2-hydroxyethyl) piperazin-1-yl]ethanesulfonic acid (HEPES) or 20 mM 2-(N-morpholino) ethanesulfonic acid (MES) buffer, 5 mM calcium chloride (CaCl₂)), and 2 mM potassium dihydrogen phosphate (KH₂PO₄). Additional ions may be included in ‘experimental’ mSBF solutions as shown in Tables 2 and 3. For example, the additional ions may include sodium metasilicate, citrate, and fluoride. Some embodiments may lack metasilicate, citrate, and/or fluoride. A calcium:phosphate ratio in the mSBF may range from about 0.2 to about 5. For example, the calcium:phosphate ratio in the mSBF may be 0.2, 0.5. 1. 2.5, or 5. Table 2 shows different ionic conditions that were used for different trials 1-21 in 20 mM HEPES buffer. Table 3 shows different ionic conditions that were used for trials E1-E40 in 20 mM MES buffer (pH 5.5-6.7). In some embodiments, the coating layer is applied with by incubation over two days in ‘regular’ mSBF with 4.2 mM carbonate (Table 1), and then incubated for three days in ‘experimental’ mSBF. Further, in some embodiments, the ‘regular’ and the ‘experimental’ mSBF solutions used may have the same type of buffer (e.g., HEPES or MES). The mSBF solutions were varied to produce MCMs with improved capabilities to stabilize mRNA complexes. For example, varying citrate and silicate concentrations, and increased phosphate to calcium ratios in the mSBF solution may increase the negative charge on the MCM and improve binding to mRNA complexes. Scanning electron microscopy (SEM) images of MCMs produced with different mSBF solutions are shown in FIGS. 19-24. The MCMs of FIGS. 19-24 were produced using 2D ‘regular’ mSBF (20 mM MES (pH 5.5-6.7), 2.1 mM carbonate) followed by 3D ‘experimental’ mSBF (20 mM MES (pH 5.5-6.7), 2.1 mM carbonate) with varying calcium phosphate ratios.

TABLE 1
Base Components for mSBF (‘Regular’ mSBF Solutions)

| Component | Molarity (mM) |
|---|---|
| NaCl | 141 |
| KCl | 4 |
| MgSO₄ | 0.5 |
| MgCl₂ | 1 |
| NaHCO₃ | 4.2/2.1 |
| HEPES/MES | 20 |
| CaCl₂ | 5 |
| KH₂PO₄ | 2 |

TABLE 2
Ionic Conditions for ‘Experimental’ mSBF Solutions

| | | | | Sodium | | |
|---|---|---|---|---|---|---|
| Trial | Calcium:Phosphate | | Carbonate | Metasilicate | Citrate | Fluoride |
| # | Ratio | Buffer | (mM) | (mM) | (mM) | (mM) |
| 1 | 0.5 | HEPES | 4.2 | — | — | — |
| 2 | 0.75 | HEPES | 4.2 | — | — | — |
| 3 | 1 | HEPES | 4.2 | — | — | — |
| 4 | 1.5 | HEPES | 4.2 | — | — | — |
| 5 | 2 | HEPES | 4.2 | — | — | — |
| 6 | 2.5 | HEPES | 4.2 | — | — | — |
| 7 | 2.5 | HEPES | 2.1 | — | — | — |
| 8 | 5 | HEPES | 4.2 | — | — | — |
| 9 | 2.5 | HEPES | 4.2 | — | 5 (1 hr | — |
| | | | | | incubation) | |
| 10 | 2.5 | HEPES | 4.2 | — | 5 | — |
| 11 | 2.5 | HEPES | 4.2 | — | 5 | 1 |
| 12 | 2.5 | HEPES | 4.2 | 6.28 | 5 | 1 |
| 13 | 2.5 | HEPES | 4.2 | — | — | 1 |
| 14 | 2.5 | HEPES | 4.2 | 6.28 | — | — |
| 15 | 2.5 | HEPES | 4.2 | 6.28 | — | 1 |
| 16 | 2.5 | HEPES | 4.2 | 6.28 | — | — |
| 17 | 0.5 | HEPES | 4.2 | — | 5 | 1 |
| 18 | 0.5 | HEPES | 4.2 | 6.28 | 5 | 1 |
| 19 | 0.5 | HEPES | 4.2 | — | 5 | — |
| 20 | 0.5 | HEPES | 4.2 | — | — | 1 |
| 21 | 0.5 | HEPES | 4.2 | — | — | — |

TABLE 3
Ionic Conditions for mSBF Solutions

| | | | Sodium | Sodium | | | |
|---|---|---|---|---|---|---|---|
| | Calcium:Phosphate | | Bicarbonate | Metasilicate | Citrate | Fluoride | |
| Trial # | Ratio | Buffer | (mM) | (mM) | (mM) | (mM) | pH |
| E1 | 0.2 | MES | 2.1 | — | — | — | 5.5 |
| E2 | 0.5 | MES | 2.1 | — | — | — | 6 |
| E3 | 1 | MES | 2.1 | — | — | — | 6.3 |
| E4 | 2.5 | MES | 2.1 | — | — | — | 6.7 |
| E5 | 5 | MES | 2.1 | — | — | — | 6.7 |
| E6 | 0.2 | MES | 2.1 | — | 5 | — | 5.6 |
| E7 | 0.5 | MES | 2.1 | — | 5 | — | 5.6 |
| E8 | 1 | MES | 2.1 | — | 5 | — | 6.3 |
| E9 | 2.5 | MES | 2.1 | — | 5 | — | 6.7 |
| E10 | 5 | MES | 2.1 | — | 5 | — | 6.7 |
| E11 | 0.2 | MES | 2.1 | — | — | 1 | 5.5 |
| E12 | 0.5 | MES | 2.1 | — | — | 1 | 5.5 |
| E13 | 1 | MES | 2.1 | — | — | 1 | 6.3 |
| E14 | 2.5 | MES | 2.1 | — | — | 1 | 6.7 |
| E15 | 5 | MES | 2.1 | — | — | 1 | 6.7 |
| E16 | 0.2 | MES | 2.1 | — | 5 | 1 | 5.8 |
| E17 | 0.5 | MES | 2.1 | — | 5 | 1 | 6.7 |
| E18 | 1 | MES | 2.1 | — | 5 | 1 | 6.7 |
| E19 | 2.5 | MES | 2.1 | — | 5 | 1 | 6.7 |
| E20 | 5 | MES | 2.1 | — | 5 | 1 | 6.7 |
| E21 | 0.2 | MES | 2.1 | 5 | — | — | 5.5 |
| E22 | 0.5 | MES | 2.1 | 5 | — | — | 5.5 |
| E23 | 1 | MES | 2.1 | 5 | — | — | 5.5 |
| E24 | 2.5 | MES | 2.1 | 5 | — | — | 5.5 |
| E25 | 5 | MES | 2.1 | 5 | — | — | 5.5 |
| E26 | 0.2 | MES | 2.1 | 5 | 5 | — | 5.5 |
| E27 | 0.5 | MES | 2.1 | 5 | 5 | — | 5.8 |
| E28 | 1 | MES | 2.1 | 5 | 5 | — | 6 |
| E29 | 2.5 | MES | 2.1 | 5 | 5 | — | 5.9 |
| E30 | 5 | MES | 2.1 | 5 | 5 | — | 5.5 |
| E31 | 0.2 | MES | 2.1 | 5 | — | 1 | 5.5 |
| E32 | 0.5 | MES | 2.1 | 5 | — | 1 | 5.5 |
| E33 | 1 | MES | 2.1 | 5 | — | 1 | 5.5 |
| E34 | 2.5 | MES | 2.1 | 5 | — | 1 | 6 |
| E35 | 5 | MES | 2.1 | 5 | — | 1 | 5.5 |
| E36 | 0.2 | MES | 2.1 | 5 | 5 | 1 | 6 |
| E37 | 0.5 | MES | 2.1 | 5 | 5 | 1 | 6.7 |
| E38 | 1 | MES | 2.1 | 5 | 5 | 1 | 6.7 |
| E39 | 2.5 | MES | 2.1 | 5 | 5 | 1 | 6 |
| E40 | 5 | MES | 2.1 | 5 | 5 | 1 | 5.5 |

In some embodiments, the mineral coating layer may further include a dopant. Suitable dopants include halogen ions, for example, fluoride ions, chloride ions, bromide ions, and iodide ions. The dopant(s) may be added with the other components of the mSBF prior to incubating the substrate 24 in the mSBF to form the mineral coating layer 20. In one embodiment, the halogen ions include fluoride ions. Fluoride ions may be provided by fluoride ion-containing agents such as sodium fluoride. The fluoride ion-containing agent may be included in the mSBF to provide an amount of up to 100 mM fluoride ions, including from about 0.001 mM to about 100 mM, including from about 0.01 mM to about 50 mM, including from about 0.1 mM to about 15 mM, and including about 1 mM fluoride ions. A halogen-doped mineral coating layer may enhance the efficiency of delivery of the mRNA to cells. Additionally, the carbonate concentration in the mSBF may be varied to tune the size and porosity of the resulting mineral coating layer. Citrate and/or silicates may be used as additional dopants in some embodiments.

After the mineral coating layer 20 is formed on the microparticles 70, the resulting MCMs 26 may be removed from the mSBF and washed. To form a plurality of mineral coating layers, the MCMs 26 may be incubated in a second, third, fourth, etc., mSBF solutions until the desired number of mineral coating layers is achieved. During each incubation period, a new layer of mineral coating may be formed on the previous layer. After the mineral coating layer(s) 20 are formed, the composition of the mineral coating layers may be analyzed by energy dispersive X-ray spectroscopy, Fourier transform infrared spectrometry, X-ray diffractometry, and combinations thereof, for example using the peaks described in U.S. patent application Ser. No. 16/626,971 incorporated herein by reference. Suitable X-ray diffractometry peaks may be, for example, at 26° and 31°, which correspond to the (0 0 2) plane, the (2 1 1) plane, the (1 1 2) plane, and the (2 0 2) plane for the hydroxyapatite mineral phase. Particularly suitable X-ray diffractometry peaks may be, for example, at 26° and 31°, which correspond to the (0 0 2) plane, the (1 1 2) plane, and the (3 0 0) plane for carbonate-substituted hydroxyapatite. Other suitable X-ray diffractometry peaks may be, for example, at 16°, 24°, and 33°, which correspond to the octacalcium phosphate mineral phase. Suitable spectra obtained by Fourier transform infrared spectrometry analysis may be, for example, a peak at 450-600 cm⁻¹, which corresponds to O—P—O bending, and a peak at 900-1200 cm⁻¹, which corresponds to asymmetric P—O stretch of the PO₄³⁻ group of hydroxyapatite. Particularly suitable spectra peaks obtained by Fourier transform infrared spectrometry analysis may be, for example, peaks at 876 cm⁻¹, 1427 cm⁻¹, and 1483 cm⁻¹, which correspond to the carbonate (CO₃²⁻) group. The peak for HPO₄²⁻ may be influenced by adjusting the calcium and phosphate ion concentrations of the SBF used to prepare the mineral coating layer. For example, the HPO₄²⁻ peak may be increased by increasing the calcium and phosphate concentrations of the SBF used to prepare the mineral coating layer. Alternatively, the HPO₄²⁻ peak may be decreased by decreasing the calcium and phosphate concentrations of the SBF. Another suitable peak obtained by Fourier transform infrared spectrometry analysis may be, for example, a peak obtained for the octacalcium phosphate mineral phase at 1075 cm⁻¹, which may be influenced by adjusting the calcium and phosphate ion concentrations in the simulated body fluid used to prepare the mineral coating layer. For example, the 1075 cm⁻¹ peak may be made more distinct by increasing the calcium and phosphate ion concentrations in the simulated body fluid used to prepare the mineral coating layer. Alternatively, the 1075 cm⁻¹ peak may be made less distinct by decreasing the calcium and phosphate ion concentrations in the simulated body fluid used to prepare the mineral coating layer.

Energy dispersive X-ray spectroscopy analysis may also be used to determine the calcium/phosphate ratio of the mineral coating layer(s) and addition of any elemental dopants (F, Si). This analysis may also be done with scanning electron microscopy alone or in conjunction with energy dispersive X-ray spectroscopy. For example, the calcium/phosphate ratio may be increased by decreasing the calcium and phosphate ion concentrations in the SBF. Alternatively, the calcium/phosphate ratio may be decreased by increasing the calcium and phosphate ion concentrations in the SBF. Analysis of the mineral coating layers by energy dispersive X-ray spectroscopy allows for determining the level of carbonate (CO₃²⁻) substitution for PO₄³⁻ and incorporation of HPO₄²⁻ into the mineral coating layers. Typically, the SBF includes calcium and phosphate ions in a ratio of from about 10:1 to about 0.2:1, including from about 2.5:1 to about 1:1.

Further, the morphology of the mineral coating layers may be analyzed by scanning electron microscopy. For example, scanning electron microscopy may be used to visualize the morphology of the resulting mineral coating layers. The morphology of the mineral coating layers may be, for example, a spherulitic microstructure, a plate-like microstructure, and/or a net-like microstructure. Suitable average diameters of the spherulites of a spherulitic microstructure may be, for example, from about 2 μm to about 42 μm. Particularly suitable average diameters of the spherulites of a spherulitic microstructure may be, for example, from about 2 μm to about 4 μm. In another embodiment, particularly suitable average diameters of the spherulites of a spherulitic microstructure may be, for example, from about 2.5 μm to about 4.5 μm. In another embodiment, particularly suitable average diameters of the spherulites of a spherulitic microstructure may be, for example, from about 16 μm to about 42 μm.

Suitable microparticle sizes may range from about 0.2 μm to about 100 μm in diameter, including from about 1 μm to about 100 μm. Microparticle diameters may be measured by methods known to those skilled in the art such as, for example, measurements taken from microscopic images (including light and electron microscopic images), filtration through a size-selection substrate, and the like.

The nanostructure morphology of the mineral coating layer(s) may be analyzed by scanning electron microscopy. For example, scanning electron microscopy can be used to visualize the nanostructure morphology of the resulting mineral coating layer(s). The morphology of the resulting mineral coating layer(s) can be, for example, plate-like nanostructures. With a plate-like microstructure, the mineral coating layers may include plates having an average diameter of from about 100 nm to about 1500 nm and an average pore size ranging from about 200 nm to about 750 nm, although plate diameters and pore sizes may be smaller or larger in some aspects. In one particularly suitable embodiment, when used in a plate-like nanostructure, the mineral coating layers include calcium, phosphate, hydroxide and bicarbonate.

In other embodiments, the mineral coating layer(s) may have a needle-like microstructure, including needles that fill out the coating and do not show observable/measurable pores. Suitably, the needles may range from about 10 nanometers (nm) to about 750 nm in length. In one embodiment, when used in a needle-like nanostructure, the mineral coating layer(s) may include calcium, phosphate, hydroxide, bicarbonate, citrate, silicates and/or fluoride.

Referring to FIG. 7, an exemplary method of preparing the mineral-coated borosilicate glass vial 28 is shown. At a block 80, the method may involve abrading/etching the inner surface 30 of the glass vial 28. In some embodiments, a high-pressure sand blasting instrument (MicroBlaster) may be used to abrade the inner surface 30. Other suitable surface abrasion techniques such as etching may also be used in some embodiments. Surface etching may be performed by treatment with sodium hydroxide in some embodiments. Functional groups on the abraded surface may be exposed by incubation in a sodium hydroxide solution at an elevated temperature (block 82). Prior to application of the mineral coating layer(s) 20, the treated surface may be optionally silanized with amino or carboxy groups according to a block 84 to provide nucleation sites for the coating layer application. For instance, the treated surface of the glass vial 28 may be silanized with 3-aminopropyltriethoxysilane or carboxyethylsilanetriol sodium salt (see Example 13). The treated surface may then be mineralized in mSBF over a period of hours to days to form the mineral coating layer 20 (block 86). The above-described mSBF compositions may be used. Multiple coating layers 20 may also be applied to the glass vial surface as described above. Incubation of a sodium hydroxide treated surface in 10X mSBF (10 times the concentration of ‘regular’ mSBF (Table 1)) at pH 6.8 provided a robust mineral coating layer without any noticeable flaking. Detailed protocols for mineralization of borosilicate glass vials are provided below in Example 12.

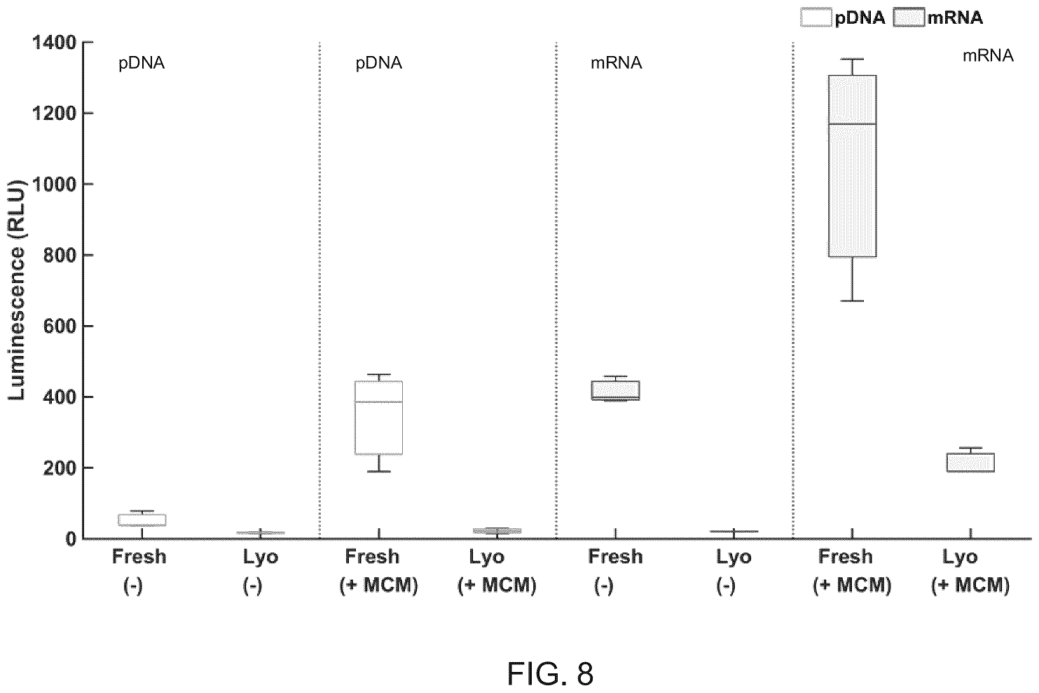

FIG. 8 shows results demonstrating the lyoprotective effect of MCMs on the mRNA complexes 12 without additional lyoprotectants (e.g., disaccharides, etc.). Experiments were carried out using mRNA 14 that encodes for Gaussia Luciferase protein to provide a detectable luminescent signal for cell transfection efficiency. Plasmid DNA (‘pDNA’) or mRNA complexes (‘mRNA’) were used fresh (‘Fresh’) or were frozen and lyophilized (‘Lyo’) with (+MCM) or without (−) MCMs. The samples were used to transfect human mesenchymal stromal cells, and cell culture media was assayed for luminescence following transfection (see Example 5). Fresh mRNA complexes had a higher transfection efficiency than lyophilized mRNA complexes with or without MCMs. However, the presence of the MCMs preserved some of the transfection efficiency that was lost due to lyophilization (compare ‘Lyo (−)’ with ‘Lyo (+MCM)’ for the mRNA data), demonstrating that the MCMs protect the mRNA complexes after lyophilization. The same lyoprotective effect was not observed for pDNA, however.

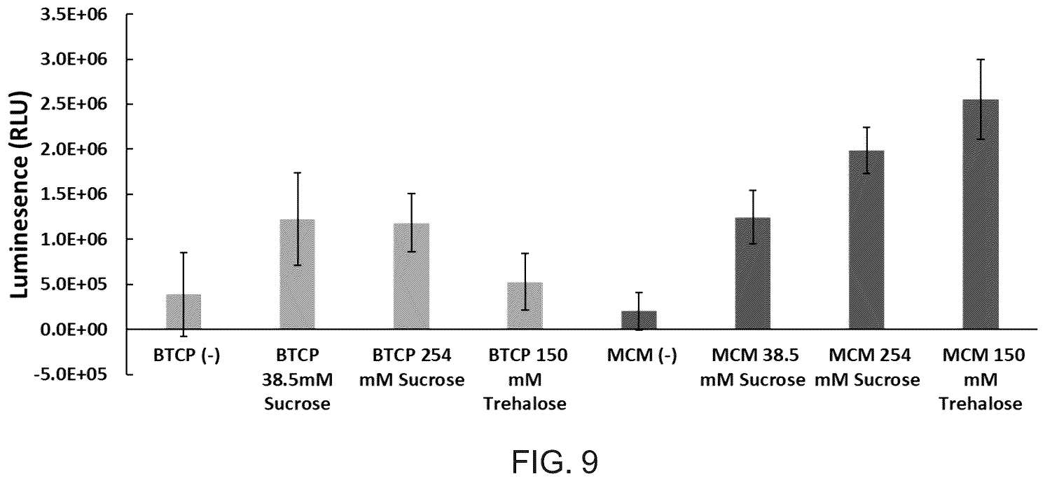

FIG. 9 shows results demonstrating the lyoprotective effect of the mineral coating layer on the mRNA complexes in the presence of disaccharide lyoprotectants. Experiments were carried out using mRNA that encodes for Gaussia Luciferase as described above with reference to FIG. 8. Lyophilized samples of mRNA complexes with β-TCP microparticles (lacking the mineral coating layer) or with MCMs (having the mineral coating layer) were used to transfect human mesenchymal stromal cells. The lyophilized samples either lacked any disaccharide lyoprotectants (−) or included sucrose (38.5 mM, 254 mM) or trehalose (150 mM). Cell culture media was assayed for luminescence as a measure of transfection efficiency and lyoprotection (see Example 5). As can be seen from FIG. 9, the mineral coating layer provides significant increases in lyoprotection in the samples containing 254 mM sucrose and 150 mM trehalose (compare ‘BTCP 254 mM sucrose’ with ‘MCM 254 mM sucrose’, and ‘BTCP 150 mM trehalose’ with ‘MCM 150 mM trehalose’). The mineral coating layer thus appears to enhance the lyoprotective effect of sucrose and trehalose disaccharides. Notably, optimal transfection efficiency was observed with the combination of MCMs with 150 mM trehalose as a lyoprotectant.

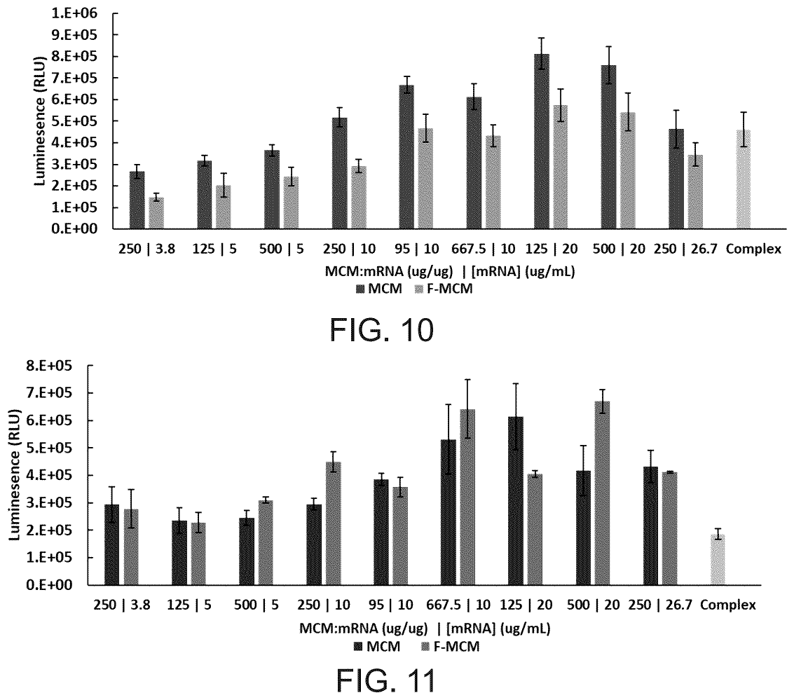

Results showing an optimization of conditions for MCM binding to mRNA complexes are shown in FIGS. 10-11. FIG. 10 shows optimal conditions for MCM binding to mRNA lipid-nanoparticle complexes, and FIG. 11 shows optimal conditions for MCM binding to mRNA-lipopolyplex complexes. MCMs and mRNA complexes were incubated under varying MCM:mRNA mass ratios (microgram (μg)/(μg)) and mRNA concentrations (in μg/milliliter (mL)). As above, the experiments were conducted using mRNA encoding for Gaussia Luciferase to provide a luminescent signal for transfection. The MCMs and mRNA complexes were used to transfect human mesenchymal stromal cells, and luminescence resulting from Gaussia Luciferase expression was used to quantify transfection efficiency of the samples. For both lipid nanoparticles (FIG. 10) and lipopolyplexes (FIG. 11), an MCM:mRNA mass ratio of 125 and an mRNA concentration of 20 μg/mL were found to provide the best transfection results (see Example 4 for additional experimental details).

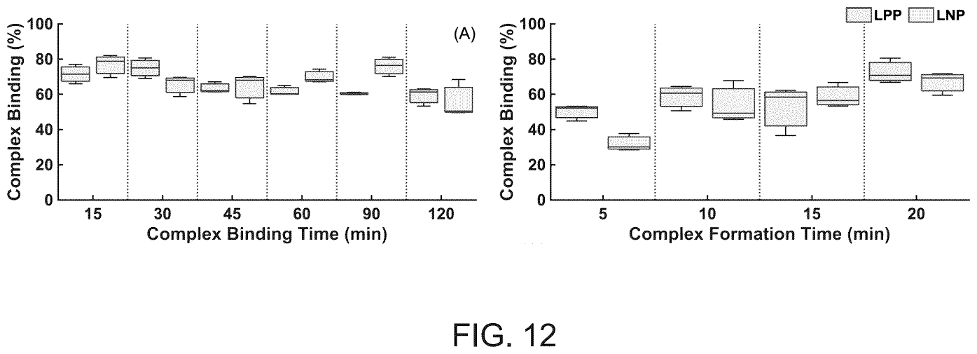

Results demonstrating the influence of complex binding time and complex formation time on binding between MCMs and mRNA complexes are shown in FIG. 12. Experiments were conducted for both mRNA-lipopolyplex complexes (‘LPP’) and mRNA-lipid nanoparticle complexes (‘LNP’). For these experiments, fluorescently labeled mRNA and complexing agents were used to form the complexes (see Example 6). After binding to the MCMs, the MCMs were centrifuged and binding was quantified using the residual fluorescence signal of the supernatant compared to the fluorescence signal of mRNA complexes alone (see Example 7 for experimental details). The results of FIG. 12 indicate that complex binding time and complex formation time minimally influence binding between MCMs and mRNA complexes.

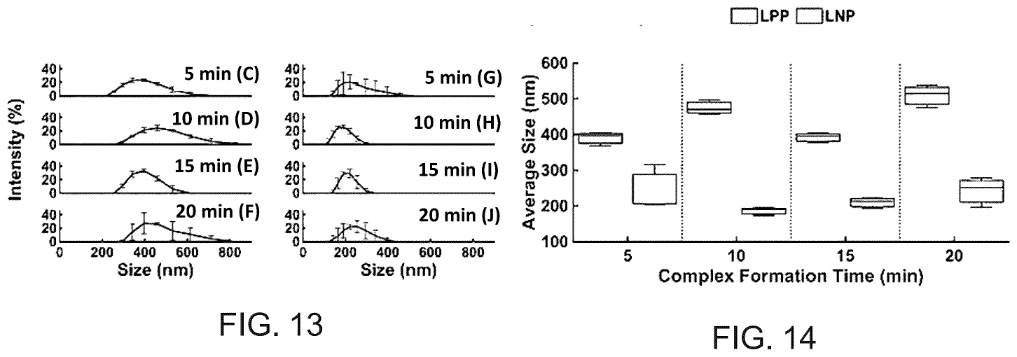

FIGS. 13-14 show changes in mRNA complex sizes with varying complexation incubation times for both mRNA-lipopolyplex complexes (‘LPP’) and mRNA-lipid nanoparticle complexes (‘LNP’). Complex sizes were quantified using dynamic light scattering measurements (see Example 7). A trend indicating increasing complex size with increasing incubation time was observed for the LPP samples. For instance, average particle size increased from around 400 nm to around 500 nm as complexation formation time increase from 5 minutes to 20 minutes for the LPP samples. A clear trend was not observed for the LNP samples over the incubation time period of 5 to 20 minutes.

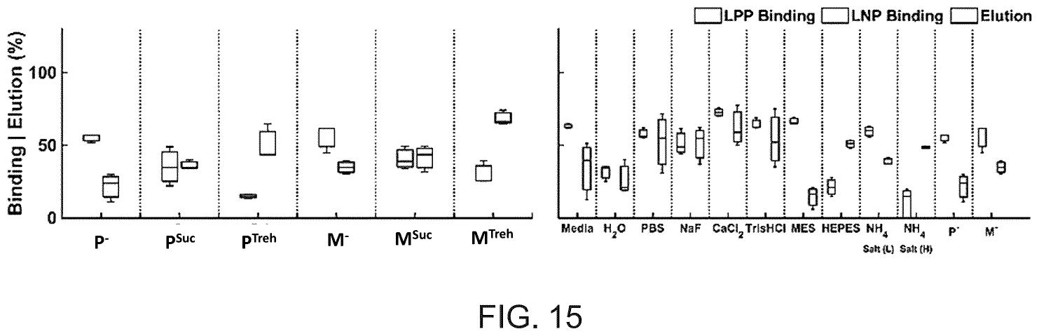

FIG. 15 demonstrates that mRNA complex binding to MCMs may be improved using solution conditions inspired by hydroxyapatite (HAP) chromatography, as HAP is similar in composition to the mineral coating layers disclosed herein. Binding of mRNA-lipopolyplex complexes (LPP) and mRNA-lipid nanoparticle complexes (LNP) to MCMs was measured in various solutions inspired by HAP chromatography including low serum media (‘media’), water (‘H₂O’), phosphate buffered saline (‘PBS’), sodium fluoride (‘NaF’), calcium chloride (‘CaCl₂)’), tris(hydroxymethyl)aminomethane hydrochloride (‘TrisHCl’), MES, HEPES, or elution buffer (25 mM ammonium phosphate (NH₄)₃PO₄, 12.5 mM ammonium carbonate ((NH₄)₂CO₃). These were compared to the excipients from the COVID-19 vaccines from Pfizer (‘P’) or Moderna (‘M’) without (−) or with sucrose (‘Suc’) or trehalose (‘Treh’) lyoprotectants. P-indicates the Pfizer excipients without lyoprotectants, M indicates the Moderna excipients without lyoprotectants, P^{Suc} indicates the Pfizer excipients with sucrose, M^{Suc} indicates the Moderna excipients with sucrose, P^{Treh} indicates the Pfizer excipients with trehalose, and M^{Treh} indicates the Moderna excipients with trehalose (see Example 9). Elution was measured by treatment with 400 mM: 200 mM ammonium phosphate:ammonium carbonate to displace the complexes. Elution buffers may further include a strong calcium binder such as a calcium binding peptide in order to prevent readsorption of the mRNA-LPP or mRNA-LNPs to the MCMs. Subtle enhancements in mRNA complex binding was observed over the Pfizer and Moderna excipients (without lyoprotectants) in the LPP samples in MES and calcium chloride, for example.

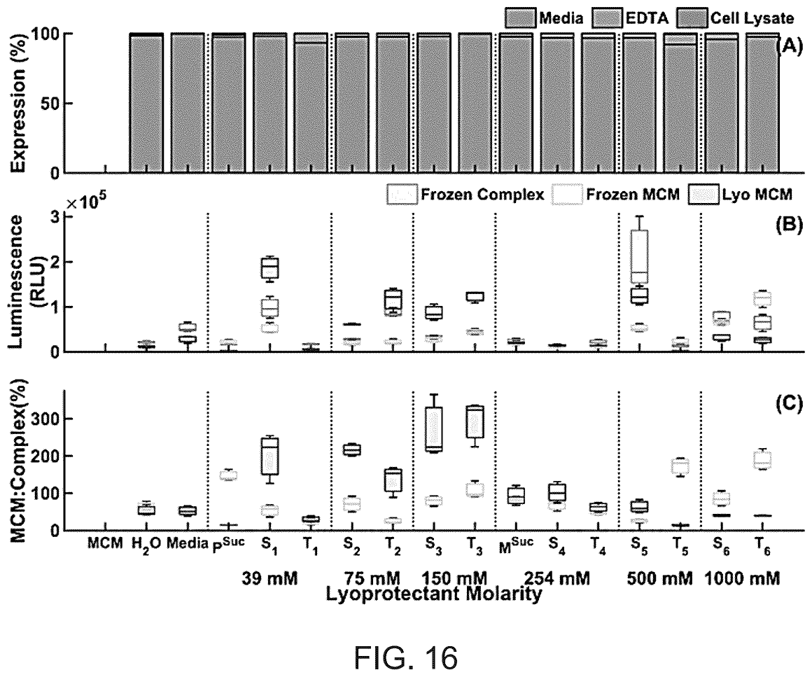

Turning to FIG. 16, Applicant has demonstrated that disaccharides improve the lyoprotective effect of MCMs on mRNA complexes. For these experiments, mRNA encoding Gaussia Luciferase was used to provide a luminescence signal for transfection. Frozen mRNA complexes (‘Frozen Complex’), frozen MCMs with mRNA complexes (‘Frozen MCM’), and lyophilized MCMs with the mRNA complexes (‘Lyo MCM’) in the presence of varying concentrations (39 mM to 1000 mM) of trehalose or sucrose were used for transfection of human mesenchymal stromal cells (see Example 8). FIG. 16A demonstrates that most of the luminescence signal used to measure expression comes from the media. Referring to FIGS. 16B and 16C, P^{Suc} represents samples containing the Pfizer excipients with the indicated concentration of sucrose (39 mM), M^{Suc} represents samples containing the Moderna excipients with the indicated concentration of sucrose (254 mM), S1-S6 indicate samples containing the indicated concentration of sucrose, and T1-T6 indicate samples containing the indicated concentration of trehalose. In FIG. 16C, MCM:Complex is the ratio of transfection (luminescence) for samples with MCMs and complexes to transfection (luminescence) of the complexes alone. As shown, the disaccharides provided enhancements in the lyoprotective effect of the MCMs, with the greatest enhancement over the Pfizer and Moderna excipients being observed with 150 mM trehalose.

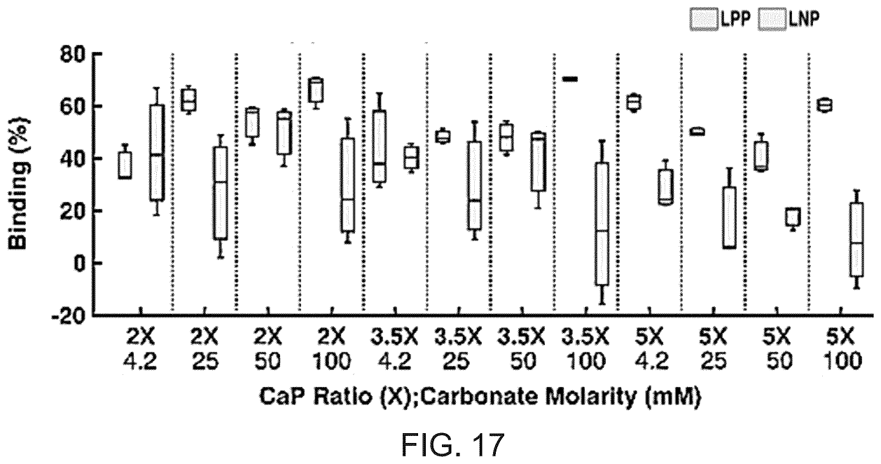

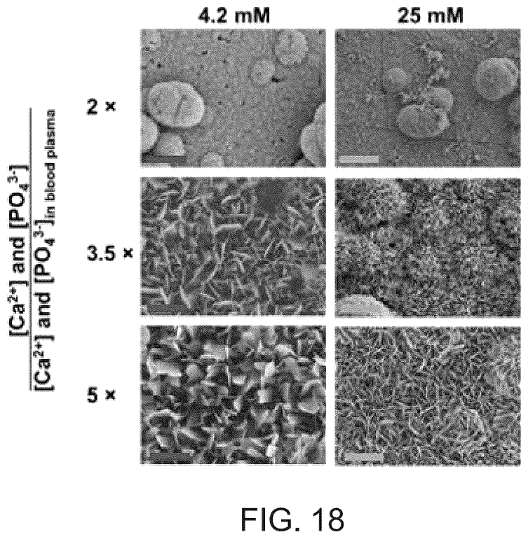

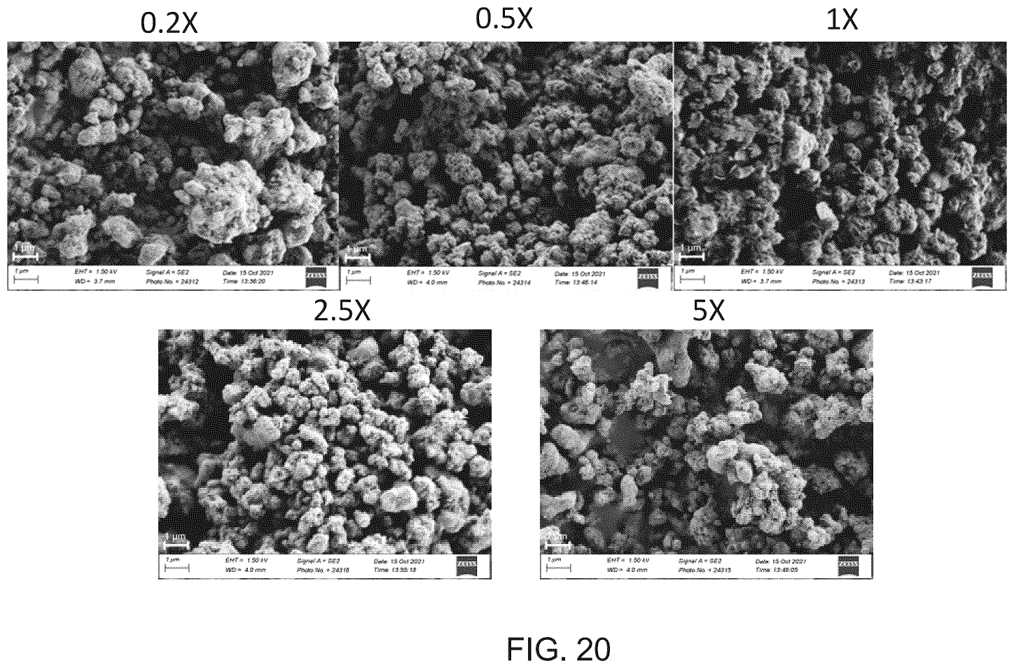

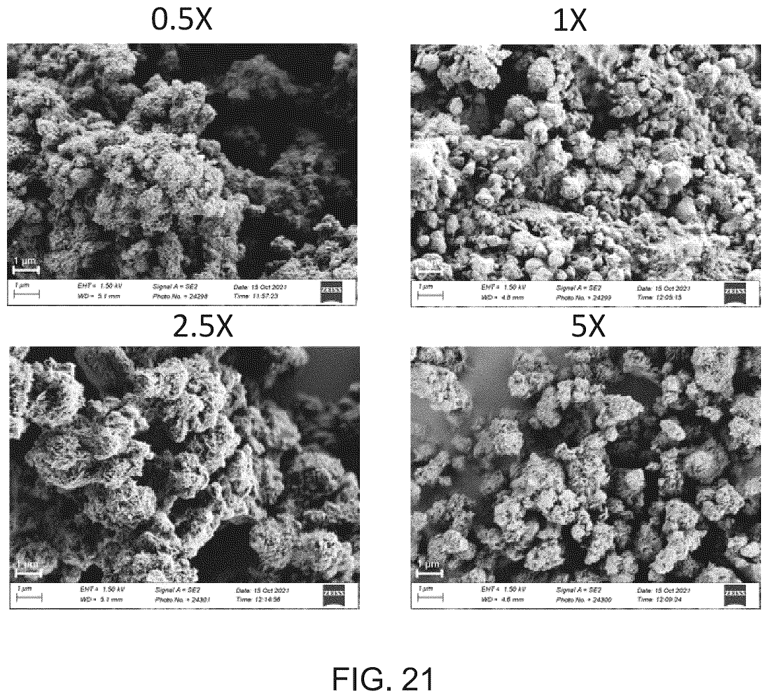

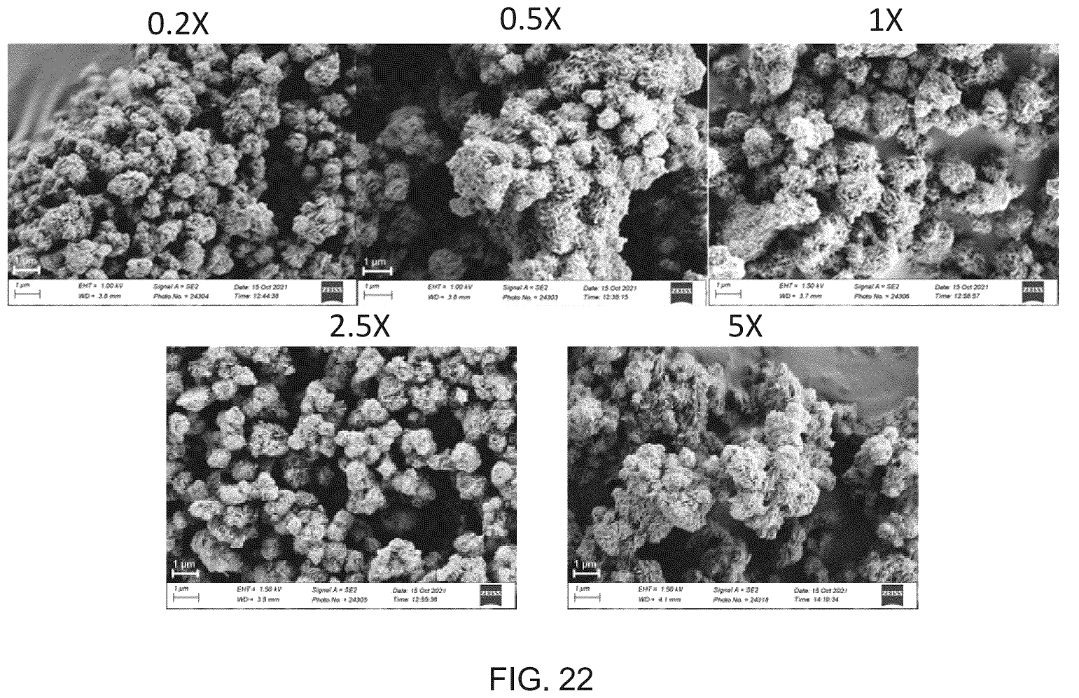

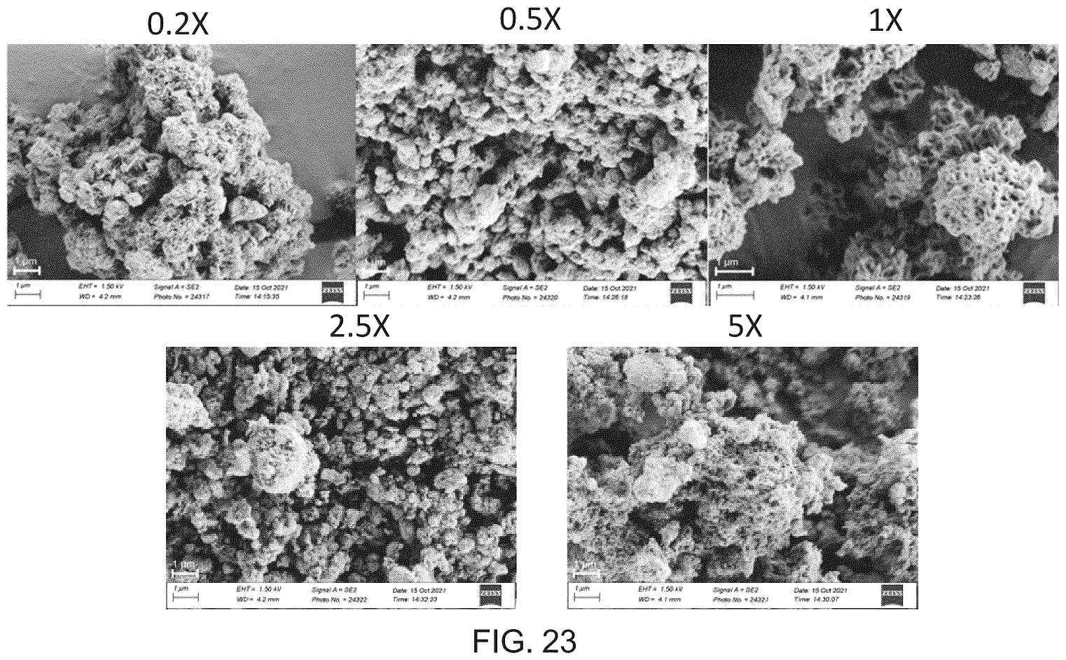

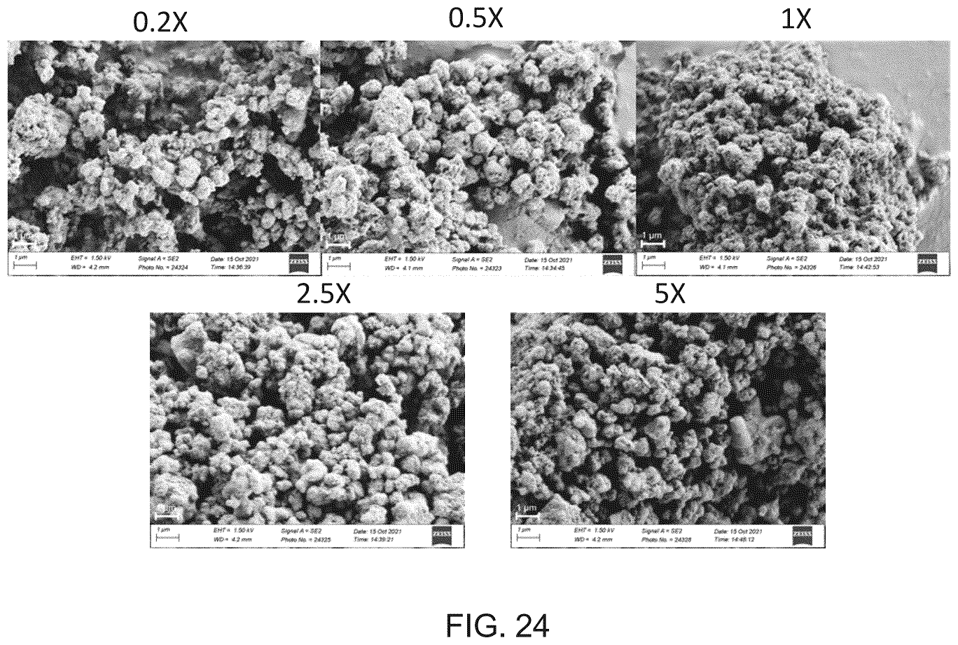

As shown in FIG. 17, the binding of mRNA-lipopolyplex complexes (LPP) and mRNA-lipid nanoparticle complexes (LNP) to MCMs may be influenced by adjusting the calcium/phosphate (CaP) ratio and the carbonate concentration in the mSBF. The calcium, phosphate, and carbonate concentrations in the mSBF also influence the morphology of the mineral coating layer. Binding was determined using a fluorescent tag on the mRNA and measuring the fluorescence of the supernatant after centrifuging the MCMs compared to the fluorescence of mRNA alone (see Example 7). FIG. 18 shows the change in morphology in the mineral coating layer at different carbonate concentrations (4.2 mM and 25 mM) and different calcium and phosphate concentrations relative to calcium and phosphate concentrations in blood plasma ([Ca²⁺] and [PO₄³⁻]/[Ca²⁺] and [PO₄³⁻] in blood plasma) in the mSBF. For instance, at a carbonate concentration of 4.2 mM, the mineral coating layer morphology transitions from spherulite-like to plate-like as the ratio [Ca²⁺] and [PO₄³⁻]/[Ca²⁺] and [PO₄³⁻] in blood plasma increases from 2 to 5. FIG. 19-24 shows scanning electron microscopy images of MCMs produced with undoped (FIG. 19), citrate doped (FIG. 20), fluoride doped (FIG. 21), citrate-fluoride doped (FIG. 22), silicate doped (FIG. 23) or citrate-silicate doped (FIG. 24) mSBF having varying calcium:phosphate ratios (0.2×, 0.5×, 1×, 2.5×, and 5×).

The mRNA-based therapeutic compositions and the reconstituted mRNA complexes may be used to treat conditions in which the mRNA will allow for expression of a peptide or protein encoded by the mRNA after delivery to a subject. The peptide or protein may be needed by the subject for many reasons as those of skill in the art will appreciate. Thus, methods of inducing an immune response in a subject comprising administering the mRNA-based therapeutic composition to a subject are provided herein. In these methods the mRNA may encode an antigen and the antigen may be derived from a microorganism, such as a pathogen. Those of skill in the art will appreciate the antigen could be a SARS-COV-2 antigen, Influenza, HIV, malaria or antigen from another pathogenic organism. Methods of supplying a protein needed by a subject, such as an mRNA encoding a protein for which the subject has insufficient levels or that could drive an immune response in a particular direction. Cytokines, antibodies or other proteins known to those of skill in the art could be delivering to alter an immune response in a subject. For example, a mRNA encoding an immunostimulatory or immunosuppressive protein could be administered to a subject with a immune refractive cancer or an autoimmune disease respectively.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc. As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like.

While the claims provided herein are directed to methods of treating a subject, both human and non-human subjects are envisioned. In addition, use of the compositions provided herein as medicaments for uses in therapy or for treating disease are also provided herein. Use of the compositions provided herein in the manufacture of a medicament for the treatment of a disease or condition are also encompassed.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

The following examples are meant only to be illustrative and are not meant as limitations on the scope of the invention or of the appended claims.

### EXAMPLES

### Example 1: Plasmid DNA Cloning and Synthesis

A commercially available plasmid (C9401 Promega, Madison, WI) with a T7 promoter site and cytomegalovirus promotor for mammalian expression was used as a template for mRNA synthesis. A synthetic gene sequence was ordered (Integrated DNA Technologies, Coralville, IA) encoding the 5′ and 3′ untranslated regions (UTRs) for the beta-globin gene (Bg; GeneID 3403) with a intervening cloning site and 20 base pair (bp) 5′ and 3′ overlaps with the cloning plasmid. This gene sequence was inserted into the plasmid just downstream of the T7 promoter using Gibson Assembly (E2611S; New England Biolabs, Ipswich, MA) with a 5-fold excess of insert to plasmid. The plasmids were linearized with restriction enzymes (AfeI, AsiSI; New England Biolabs, Ipswich, MA) and a custom synthetic Gaussia Luciferase (GenBank: AY015993.1) gene with 5′ and 3′ overlaps at the insert sites was cloned into the plasmid using Gibson assembly. All plasmids were transformed into chemically competent E. coli (C4040-06; Thermofisher Scientific, Waltham, MA). E. coli were expanded and plasmids were harvested using a plasmid mini-prep kit (K0503; Thermofisher Scientific, Waltham, MA). All nucleic acids used in this study were quantified using 260 nm/280 nm and 260 nm/230 nm as defined on a multi-channel spectrophotometer (Take3 plate, Synergy HTX plate reader, Biotek, Winooski, Vermont). Plasmids were sequenced using Sanger Sequencing at a University of Wisconsin Madison core facility and verified against expected sequences using National Center for Biotechnology Information (NCBI) Basic Logical Assignment Search Tool (BLAST). SEQ ID NO: 1 was used for the beta globin insert (see Table 4). SEQ ID NO: 2 was used for the Gaussia Luciferase insert. SEQ ID NO: 3 and SEQ ID NO: 4 were used for first and second forward primers, respectively. SEQ ID NO: 5 and SEQ ID NO: 6 were used for first and second reverse primers, respectively.

### Example 2: mRNA Synthesis

mRNA for Gaussia Luciferase were synthesized from linearized polymerase chain reaction (PCR) DNA templates. Templates were prepared using forward primers for the T7 polymerase site and reverse primers at the end of the 3′ UTR for the beta globin gene with an appended 120 nucleotide polythymidylic acid (polydT) tail. PCR was performed using a high-fidelity DNA polymerase master mix (M0532, New England Biolabs, Ipswich, MA) with a 1:3 ratio of forward to reverse primers. mRNA were synthesized from up to 0.2-1 μg of DNA template, 2 mM nucleotides, 8 mM of a 3′-O-Me-m⁷G(5′)ppp(5′)G RNA Cap Structure Analog (ARCA; S1411, New England Biolabs, Ipswich, MA), 1 unit/microliter (μl) RNase inhibitor, 1.25 mM each of chemically modified nucleotides (5 m cytidine-5′-triphosphate (5mCTP), pseudouridine-5′-triphosphate (pseudoUTP), Trilink Biotechnologies. San Diego, CA) and 100 units of T7 polymerase buffer for 2 hours at 37° C. mRNA was purified using spin columns (T2040, New England Biolabs, Ipswich, MA), quantified and verified for size and integrity on a 1% bleach 1% agarose gel or Bioanalyzer (Agilent, Santa Clara, Ca). SEQ ID NO: 7 (T7 Polymerase Forward Primer) and SEQ ID NO: 8 (PolydT Tail (120) Reverse Primer) were used for the mRNA synthesis primers. Table 5 provides details regarding the PCR protocol used.

TABLE 4
Nucleic Acid Sequences

| SEQ ID | |
|---|---|
| NO. | Sequence |
| 1 | ctagagtattaatacgactcactatagggctagcgacatttgcttctgacacaactgtgttcactagcaa |
| | cctcaaacagacaccagcgctacagtgtacaatgacgatcagcgatcgctcgctttcttgctgtccaatt |
| | tctattaaaggttcctttgttccctaagtccaactactaaactgggggatattatgaagggccttgagca |
| | tctggattctgcctaataaaaaacatttattttcattgcaagtttaaacgaattcgggctcggtacccgg |
| | ggatc |
| 2 | ctgtgttcactagcaacctcaaacagacaccatgggagtcaaagttctgtttgccctgatctgcatcgctgtgg |
| | ccgaggccaagcccaccgagaacaacgaagacttcaacatcgtggccgtggccagcaacttcgcga |
| | ccacggatctcgatgctgaccgcgggaagttgcccggcaagaagctgccgctggaggtgctcaaagagat |
| | ggaagccaatgcccggaaagctggctgcaccaggggctgtctgatctgcctgtcccacatcaagtgcacg |
| | cccaagatgaagaagttcatcccaggacgctgccacacctacgaaggcgacaaagagtccgcacaggg |
| | cggcataggcgaggcgatcgtcgacattcctgagattcctgggttcaaggacttggagcccatggagcagtt |
| | catcgcacaggtcgatctgtgtgtggactgcacaactggctgcctcaaagggcttgccaacgtgcagtgt |
| | tctgacctgctcaagaagtggctgccgcaacgctgtgcgacctttgccagcaagatccagggccaggtgg |
| | acaagatcaagggggccggtggtgactaagctcgctttcttgctgtccaatttctatta |
| 3 | tctctccacaggtgtccactc |
| 4 | cacaactgtgttcactagcaacc |
| 5 | cctttcgggctttgttagcag |
| 6 | ggcagaatccagatgctcaag |
| 7 | gctagagtattaatacgactcactataggg |
| 8 | tttttttttttttttttttttttttttttttttttttttttttttttttttttttttttttttttttttttttttttttttt |
| | ttttttttttttttttttttttttttttttttttttttttgcaatgaaaataaatgtttttt |

TABLE 5
High Fidelity PCR Protocol

| Temperature | Time (2) | Number of Cycles |
|---|---|---|
| 94° C. | 120 | 1 |
| 98° C. | 5 | 35 |
| 60° C. | 15 | 35 |
| 4° C. | — | 1 |

### Example 3: Mineral-Coated Microparticle (MCM) and Mineral-Coated Plate Synthesis

Beta tricalcium phosphate (BTCP) micropowder (Plasma Biotal Limited, Maharashtra, India) was suspended in modified simulated body fluid (mSBF) solutions at 3 mg/mL. The suspensions were rotated at 37° C. for 24 hrs, at which point the microparticles were centrifuged at 2,000 g for 2 minutes, and the supernatant decanted and replaced with fresh mSBF daily for 5 days. For alternative compositions, MCMs were synthesized with the same base mSBF solution (Table 1) for 2 days then the experimental mSBF for an additional 3 days. MCMs were washed two times with 50 mL deionized water, filtered through a 40 micrometer (μm) pore cell strainer, suspended in 10 mL distilled water, frozen in liquid nitrogen, and lyophilized for 48 hrs. The lyophilized MCMs were then analyzed for nanotopography and calcium release as previously described. Additional MCM compositions were created by varying amounts of citric acid (5 mM), NaF (1 mM), sodium metasilicate (5 mM) and varying the calcium to phosphate ratio (0.2-5×) with a fixed calcium molarity (5 mM). MCMs were also created with incubation in 5 mM citric acid for 1 hour then washed and lyophilized as described above. Mineral-coated plates with varying calcium/phosphate ratios (2.5, 3.5, 5×) and carbonate molarities (4.2, 25, 50, 100 mM) were also prepared as previously described.

### Example 4: mRNA Complex and MCM Binding

Under sterile and nuclease free conditions, 100 nanograms (ng) of mRNA or pDNA was mixed with commercially available complexing agents. These were either a non-organizing lipopolyplex (MIR2250, Mirus Bio, Madison, WI), or a lipid nanoparticle (mRNA: LMRNA001; pDNA: L3000001, Thermofisher Scientific, Waltham, MA). This solution was incubated at room temperature under constant rotation for 20 minutes. Surface response design of experiments (JMP, SAS, Cary, NC) was used with two factors: 1) mass of MCMs to mRNA (μg:μg) and 2) concentration of mRNA complexes (μg/mL) to determine optimal loading conditions (see Table 6). Factors F-MCM were centered at 10 μg/mL of mRNA complexes and 250 g:μg MCMs:mRNA with log 2 steps (+1 for the surface positions and +1.41 for the axial positions). A 1 mg/mL stock solution of MCMs was diluted to create a final concentration based on the design of experiments software. MCMs and mRNA were incubated under constant rotation for 30 minutes at room temperature, centrifuged for 30 seconds at 2000 g, and the binding solutions aspirated. MCMs+mRNA complexes were then re-suspended in low serum media and added to cell culture. A final concentration of 125 μg MCMs:μg mRNA was used for all other experiments with a concentration of 20 g/mL mRNA.

TABLE 6
Surface Design of Experiments for mRNA Loading on MCMs

| | | MCM:mRNA | |
|---|---|---|---|
| | Log_2 | (μg/μg) | [mRNA] μg/mL |
| −1.41 | −1.41 | 95 | 3.8 |
| −1 | −1 | 125 | 5 |
| Center | 0 | 250 | 10 |
| 1 | 1 | 500 | 20 |
| 1.41 | 1.41 | 667.5 | 26.7 |

### Example 5: Cell Culture and Transfection Efficiency

Human mesenchymal stem cells (hMSCs) (Passage 3-6, PT-2501, Lonza, Basel, CH) were grown in 10% fetal bovine serum (FBS), 1% penicillin/streptomycin alpha minimum essential medium (50-012-PC, Corning, NY). mRNA complexes or mRNA complexes bound to MCMs were added directly to cells. Cell culture media was collected 24-48 hours after transfection. Media was mixed with 20 μM coelentarazine (Promega, Madison, WI) in PBS and assayed immediately on a plate reader (Synergy HTX, Biotek, Winooski, Vermont) at 460/40 nm. To evaluate the impact of transfection on cell metabolism, 1:10 of resazurin based reagent in cell culture media (G8080, Promega, Madison, WI) was added. Metabolic conversion of resazurin to resorufin was measured at 590 nm on a plate reader after 2 hours of incubation with cells.

### Example 6: Labeling of mRNA and Complexes

mRNA: mRNA was conjugated to a fluorescent molecule or biotin and purified using a commercially available reagent per manufacturer protocol (MIR3224, MirusBio, Madison, WI).

Complexing agents: Complexing agents were labeled with a 1:1 mixture of 1 μl/mL of CM-Dil dye (C7000, ThermoFisher Scientific, Waltham, MA) in PBS for 20 minutes at 37° C. Complexes were centrifuged at 3500 g for 5 minutes and washed twice in PBS.

Dual labeled mRNA complexes: Dual labeled mRNA complexes were bound to MCMs, lyophilized, resuspended, and transfected to hMSCs for 6 hours. Cells were fixed in neutral buffered formalin and stored in PBS. Cells were imaged by confocal microscopy.

Gold labeling: Biotin labeled mRNA was incubated with a diluted (1:50) streptavidin-conjugated gold nanoparticle (Nanoprobes, Yaphank, NY) for 1 hour at 37° C. mRNA was purified using a NAP-25 column (Sigma Aldrich) and verified by ultraviolet-visible (UV/VIS) spectrophotometry.

### Example 7: Binding Times and Dynamic Light Scattering

To determine the effect of varied complex formation time, fluorescently labeled mRNA (see Example 6) and (unlabeled) complexing agents were allowed to form complexes for 5 to 20 minutes in low serum media. Complex size was determined by dynamic light scattering (Zetasizer, Malvern Panalytical, Malvern, UK) at each time point. Complexes formed over 5 to 20 minutes were also bound to MCMs for 30 minutes. MCMs were centrifuged and the supernatant was read for residual fluorescence and compared to the fluorescence signal of mRNA complexes alone. To determine the effect of MCM binding time, complexes were formed for 20 minutes in low serum media and then bound to MCMs for 15 to 20 minutes. Residual fluorescence was assayed as described above.

### Example 8: Optimization of Lyophilization Conditions

mRNA was bound to MCMs and resuspended in the same volume as used for binding conditions in nuclease free solutions: water, 38.5 mM, 50 mM, 75 mM, 150 mM, 254 mM, 500 mM, 1000 mM D(+)-Trehalose (T9449, Millipore Sigma, Burlington, MA) or D(+)-sucrose. Samples were then flash frozen in liquid nitrogen and lyophilized overnight. Samples were resuspended in low serum media (11058021, ThermoFisher Scientific, Waltham, MA) prior to addition to cells. To determine the necessity of complexing agents, mRNA was bound to MCMs with or without complexes and lyophilized prior to transfection.

### Example 9: Binding Conditions

mRNA complexes were created for 20 minutes in solutions inspired by hydroxyapatite chromatography: low serum media, 25 mM CaCl₂), MES, NaF, HEPES, TrisHCl, PBS, or elution buffer (25 mM ammonium phosphate ((NH₄)₃PO₄): 12.5 mM ammonium carbonate ((NH₄)₂CO₃)). 25-100 mM MES and CaCl₂) and combination solutions were created. These were compared to the excipients from the COVID-19 vaccines (Pfizer (P) and Moderna (M)) with or without sugars. All solutions were pH 7.25 in nuclease free water. Residual fluorescence was measured as described above. The best solution conditions were used for transfection studies. mRNA complexes were prepared in 25 mM citrate buffer (pH 3.0) and dialyzed into low serum media, MES, PBS, or the vaccine excipients without sugars. Dialysis occurred over 4 hours with a replacement of the solutions after 2 hours. mMRNA complexes were bound to MCMs and lyophilized in water, 38.5 mM sucrose, 150 mM trehalose, or 254 mM sucrose. Samples were lyophilized overnight and transfected in hMSCs.

### Example 10: Accelerated Degradation

mRNA complexes were prepared in citrate buffer and dialyzed into MES buffer, then bound to MCMs. Samples were then lyophilized in 150 mM trehalose or in Pfizer or Moderna excipients and sealed under nitrogen. Paraffin sealed tubes in triplicate were stored at 25° C., 37° C., 50° C., 75° C., or 120° C. for 0, 12, 24, 48, 72, 144, and 240 hours. At each time point, samples were kept frozen at −80° C. alone with control samples kept at −80° C. After all samples were collected, they were resuspended and transfected in hMSCs. The Arrhenius equation (equation (1)) was used to model first order degradation kinetics (equation (2)) of the luciferase transfection signal, and to determine an accelerated shelf life condition of mRNA complexes bound to MCMs. In equations (1) and (2) below, k is the rate constant, A is a pre-exponential factor, Ea is activation energy, R is the universal gas constant, and T is temperature (in Kelvin).

$$ln ( k ) = - {E}_{a} / RT + ln ( A ) \qquad ( 1 )$$

$$ln [ A ] = {ln [ A ]}_{0} - kt \qquad ( 2 )$$

### Example 11: Mineral-Coated Plate Transfection

To determine the impact of varying mineral coatings, mRNA complexes were added to the mineral-coated wells of a 96 well plate. Combinations of 0.1% Triton-X100, 5000 units heparin, and 5000 units RNase were added to the wells to determine the protective effects of mineral coating on complex stressors. Plates were washed 2 times with PBS and hMSCs were seeded on top of the plates for bottom up transfection. Media was collected after 24 hours and assayed for luminescence as described above.

### Example 12: Mineral-Coated Vial Synthesis

Commercially available type B borosilicate glass vials (borosilicate glass vials (FS60910A 12-Fisher Scientific) were not pre-treated or were abraded with 150 μm aluminum oxide (Al₂O₃) or 50 μm silicon carbide (SiC) micropowders using a pressurized system (Microblaster, Comco, Burbank, CA). Organic residues were removed by immersion in 3:1 sulfuric acid (H₂SO₄): hydrogen peroxide (H₂O₂) for 30 minutes, rinsed in deionized water twice and allowed to dry. Functional groups were exposed by incubation in 0.5 M sodium hydroxide (NaOH) for 30-240 minutes at 95° C., rinsed in deionized water twice, and allowed to dry. Ten times (10×) mSBF, pH 6.8 was added to the vials for precursor mineralization for 1-2 days. The vials were washed twice in deionized water and replaced with 1×mSBF, pH 6.8 for 4-5 days. Mineralization in mSBF occurred over 6 days total at 37° C. under constant rotation. mSBF solutions were replenished daily. Following mineralization, glass vials were imaged with a digital camera or a flatbed scanner and shattered for electron microscopy or used for other experiments.

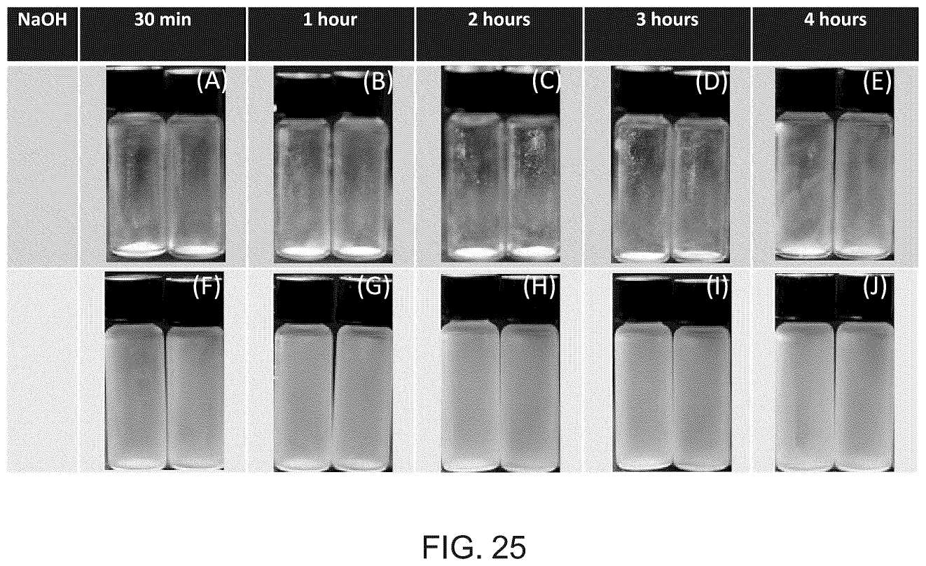

Impact of Surface Abrasion on Mineralization (Sodium Hydroxide Pretreatment Time): Glass vials were abraded with 50 micron SiC micropowders using a Microblaster (5 seconds). Control samples were not abraded. Both groups (abraded and control vials) were cleaned with piranha solution and then treated with sodium hydroxide at 100° C. for 30 minutes to 4 hours. Mineralization was carried out using 2D of 10×mSBF (pH 6.8) followed by 1×mSBF (pH 6.8) for 4 days. Increasing incubation times with sodium hydroxide improved mineralization of the glass vials (see FIG. 25).

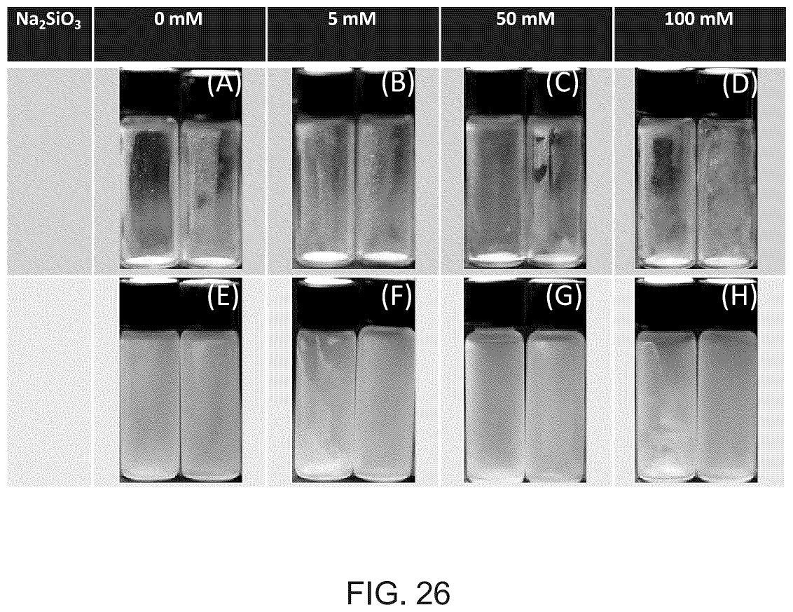

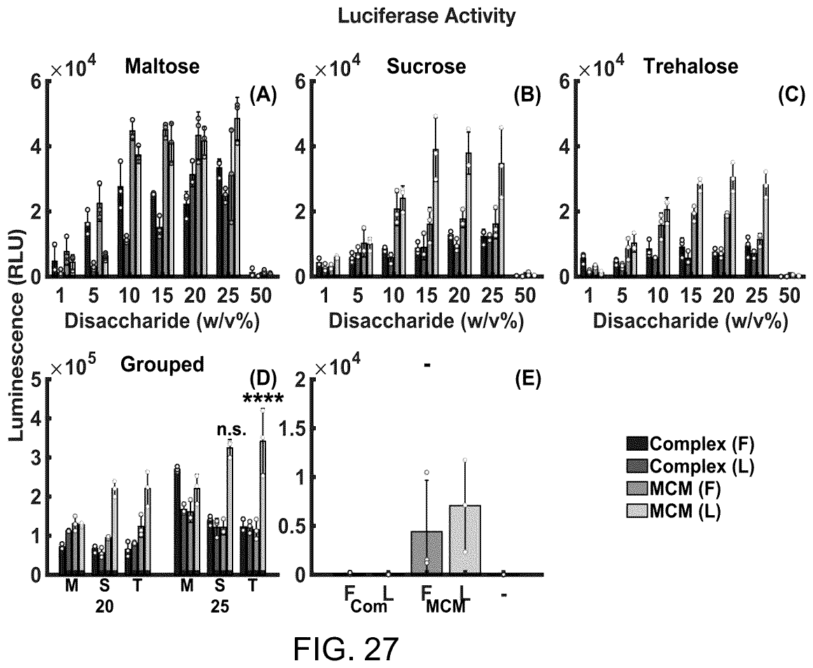

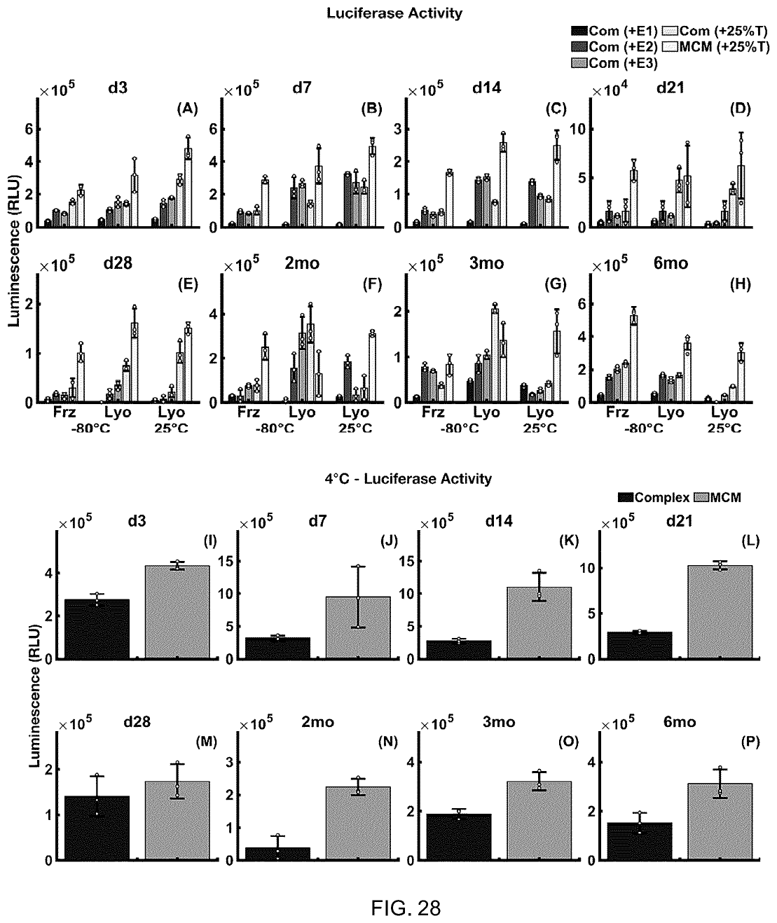

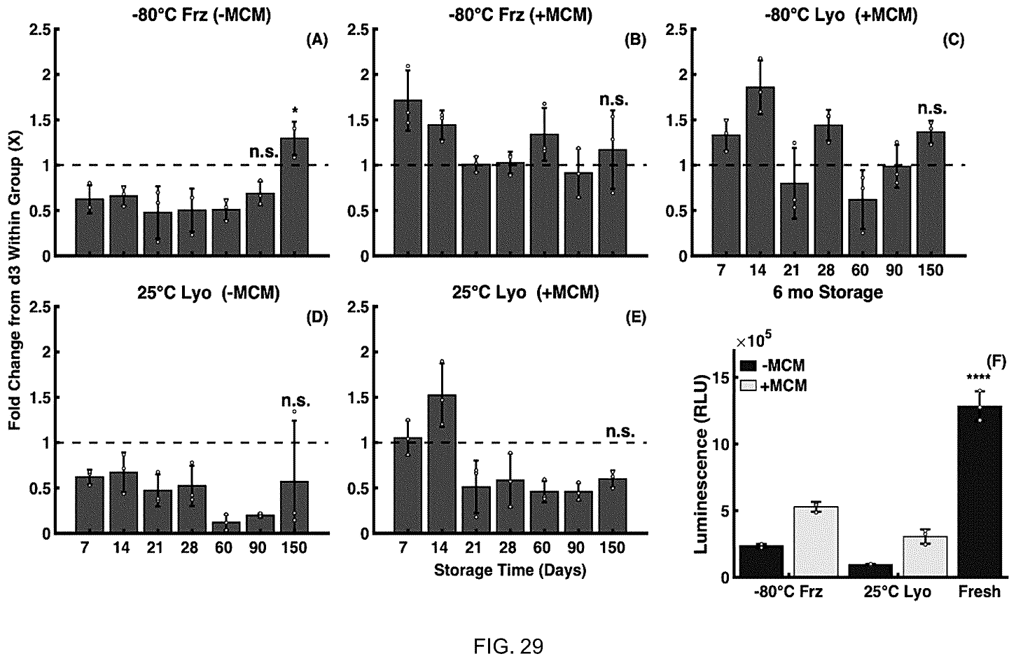

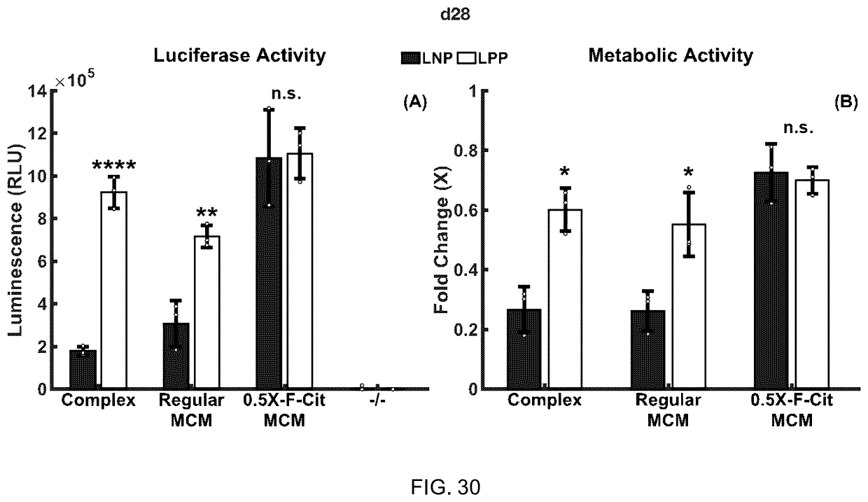

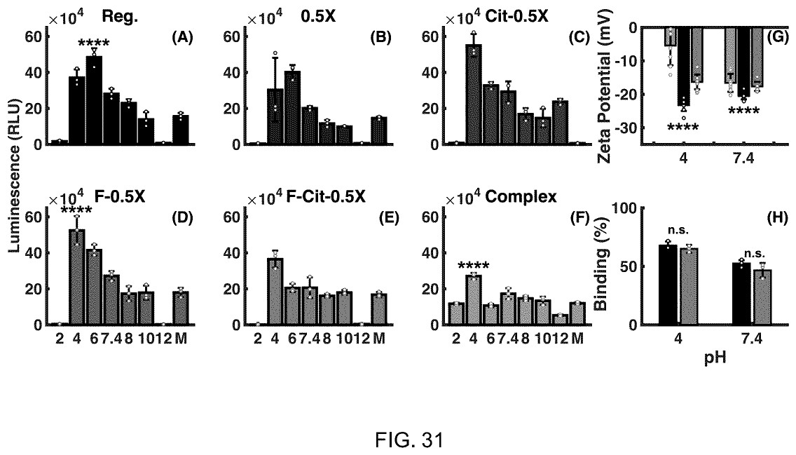

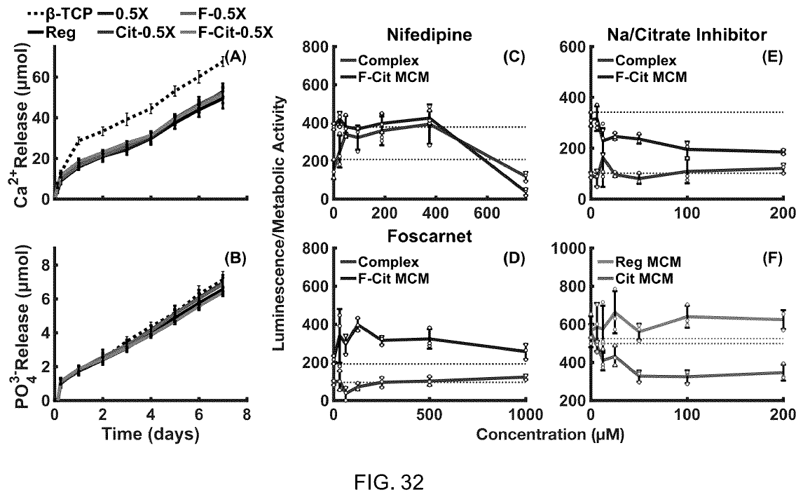

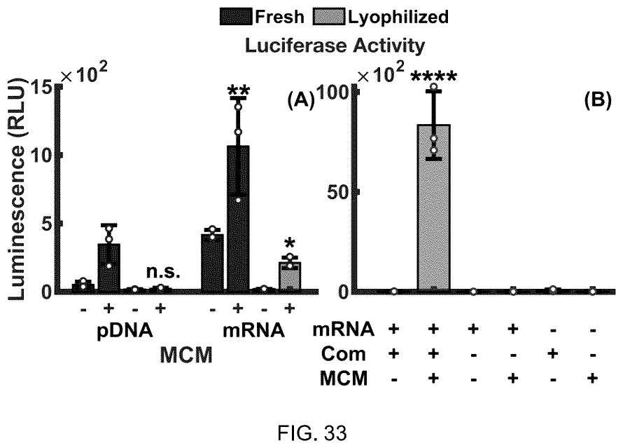

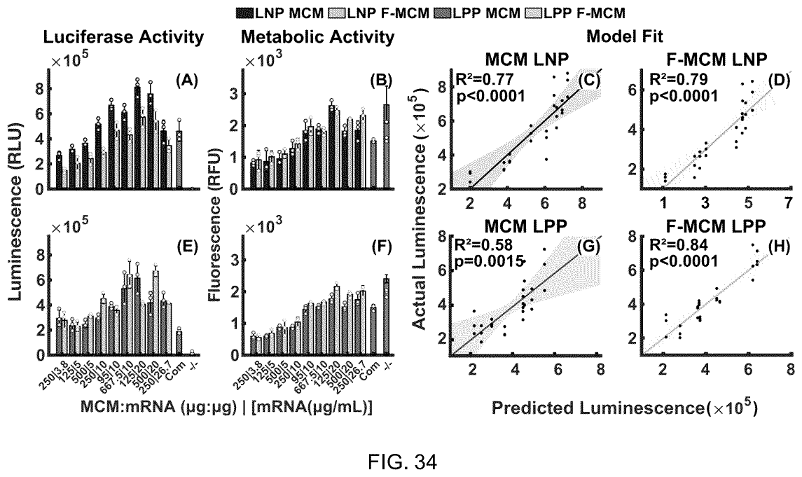

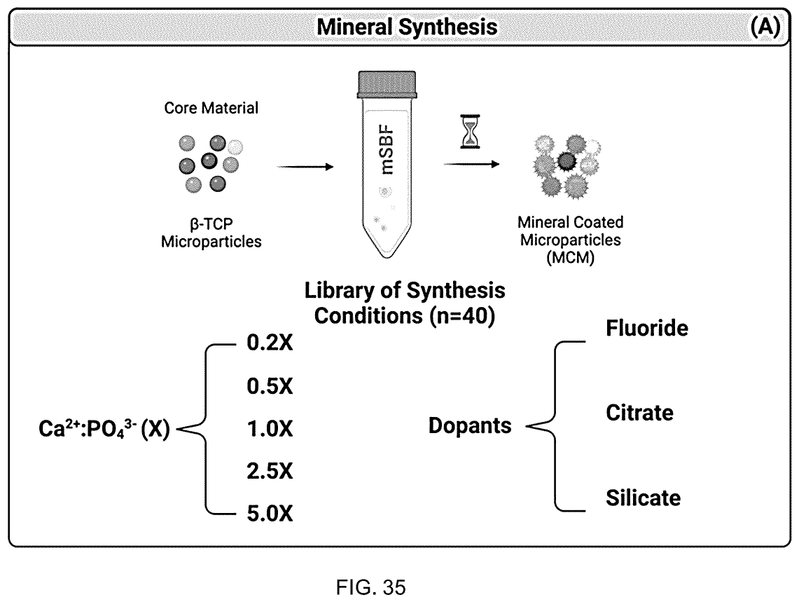

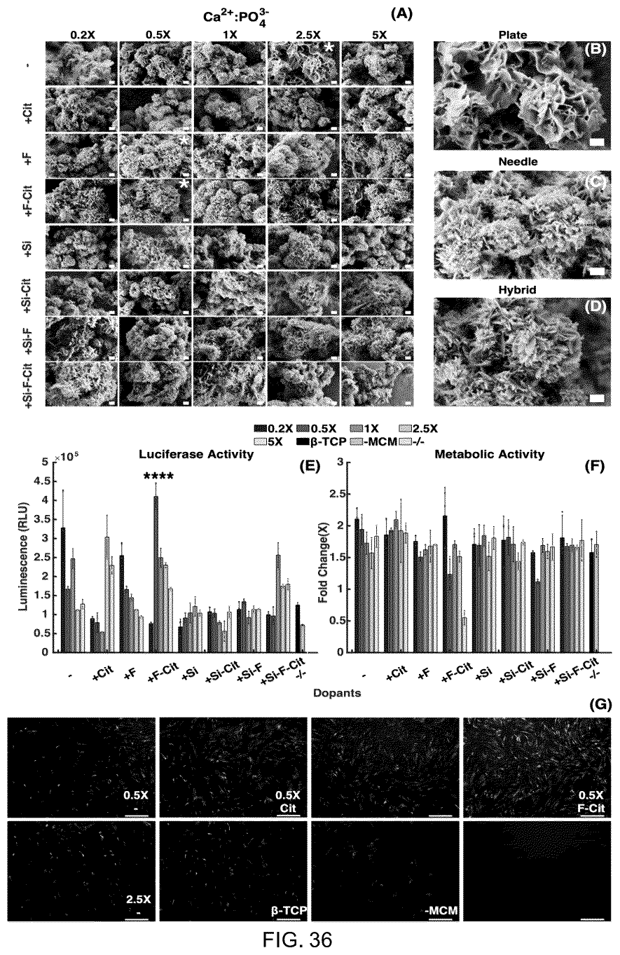

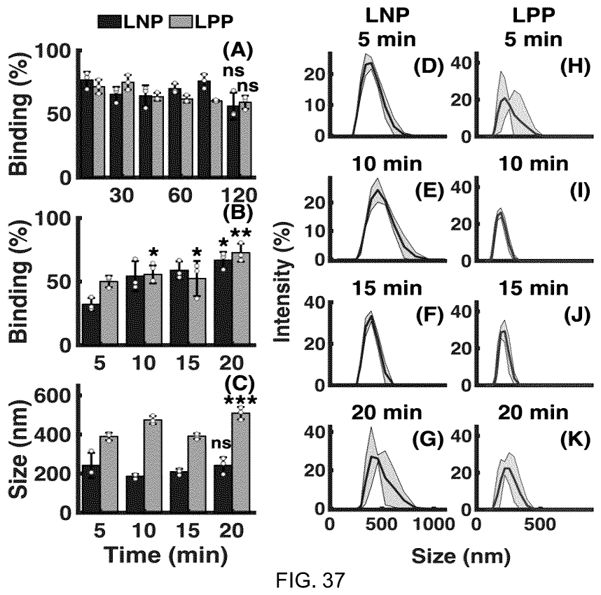

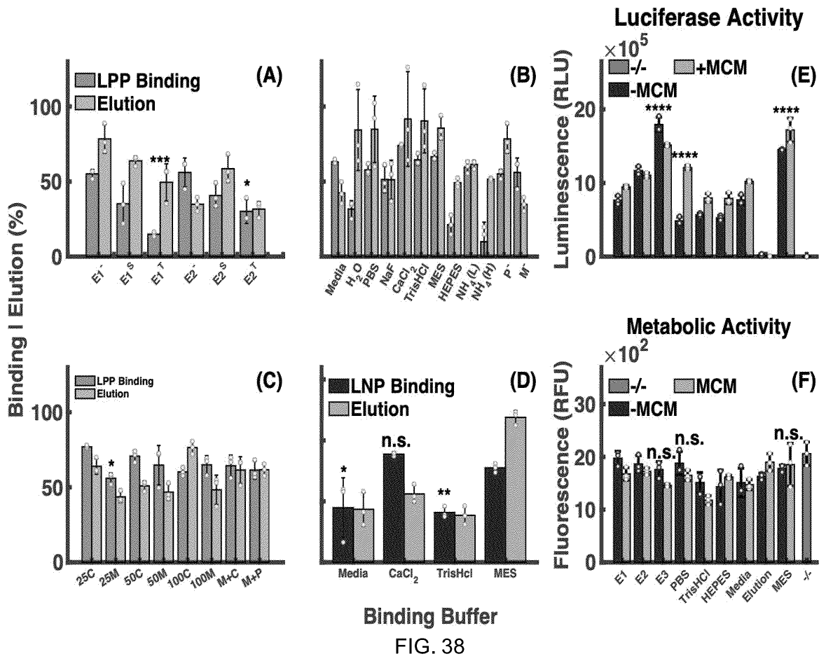

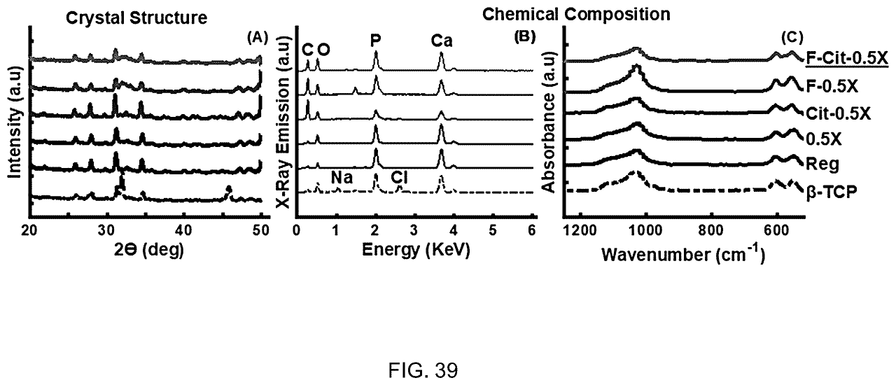

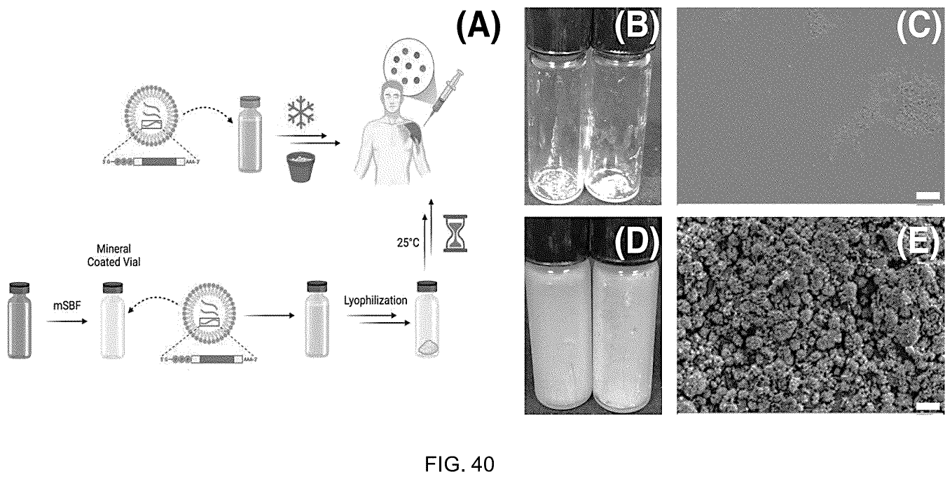

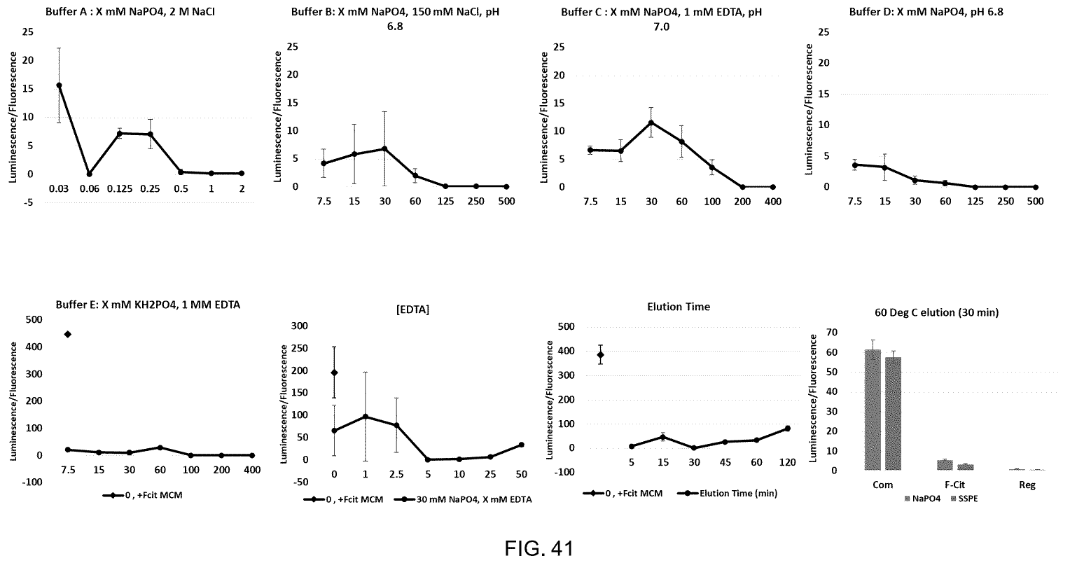

Impact of Surface Abrasion on Mineralization (Sodium Metasilicate Pretreatment Time): Glass vials were abraded with 50 micron SiC micropowders using a Microblaster (5 seconds). Control samples were not abraded. Both groups (abraded and control vials) were cleaned with piranha solution and then treated with sodium hydroxide at 100° C. for 30 min. Both groups were then treated with varying concentrations of sodium metasilicate (Na₂SiO₃) (0 mM, 5 mM, 50 mM, and 100 mM) without curing. Mineralization was carried out using 2D of 10×mSBF (pH 6.8) followed by 1×mSBF (pH 6.8) for 4 days. FIG. 26 shows pictures of both abraded (bottom) and control (top) glass vials.

### Example 13: Testing Pre-Treatments for Glass Mineralization

Multiple pre-treatment methods were tested on borosilicate glass vials to improve mineralization as described in the following:
1) Glass vials were incubated in a 1:1 mixture of H₂O₂ and 2N NaOH in a fume hood for 2 hours.
2) 1 mL SigmaCote (SL2, Sigma-Aldrich, St. Louis, MO) was added to borosilicate glass vials for 1 minute. The vials were then aspirated and washed twice in deionized water.
3) 4% (w/v) poly(D,L-lactide-co-glycolide) (PLG; Sigma-Aldrich, St. Louis, MO) was dissolved in acetone and added to the glass vials. The solution was allowed to dry overnight and then the vials were washed twice in deionized water.
4) Vials were incubated in 1×mSBF, pH 6.8 for 6 days with fresh mSBF daily.
5) Vials were soaked in 5.5 or 100 mM sodium metasilicate for 1 hour, and then treated with 2D 10×mSBF (pH 6.8), followed by treatment with 1× ‘regular’ mSBF for 4 days.
6) Silanization with amino and carboxyl groups: In order to increase the amino and carboxyl groups on the surface of the glass as sites for nucleation, the glass vials were silanized with 3-aminopropyltriethoxysilane or carboxyethylsilanetriol-sodium salt. A 2% solution of 3-aminopropyltriethoxysilane (APTS, Sigma Aldrich, St. Louis, MO) in acetone was added to glass vials for 1 hour and cured for 30 minutes at 300° C. Surfaces were rinsed with acetone and air dried. Alternatively, 25% carboxyethylsilanetriol-sodium salt (CES, Gelest, Morrisville, PA) was diluted to 4% w/v in deionized water and titrated to a pH of greater than 5.5 and less than 6.0 with glacial acetic acid. CES was added to glass vials for 24 hours at 70° C. Vials were washed twice in deionized water and cured for 30 minutes at 100° C. in air. Following silanization, 10×mSBF pH 5.5 or 6.8 was added to the vials for precursor mineralization for 1 day. The vials were washed twice in deionized water and replaced with 1×mSBF pH 6.8 daily for 5 days at 37° C.

### Example 14: Scanning Electron Microscopy

Samples (microparticles or glass shards) were placed on carbon conductive tabs (Ted Pella, Redding, CA) and sputter coated with 10 nm of platinum using a high vacuum deposition sputter coating machine (ACE, Leica, Wetzlar, Germany). Samples were imaged with scanning electron microscopy (GeminiSEM 450, Zeiss, Oberkochen, Germany) at 3-5 kilovolts (kV).

### Example 15: Disaccharide Screening and Shelf-Life Studies

MCMs were UV-sterilized and resuspended in low serum media at 1 mg/mL. mRNA complexes were bound to MCMs and resuspended in the same volume as used for binding conditions in nuclease free solutions. For MCM screening 150 mM trehalose was used prior to lyophilization. The following excipients were used for screening lyophilization conditions water, 1-50% D(+)-Trehalose (Millipore Sigma, Burlington, MA, USA), D(+)-Sucrose (Millipore Sigma, Burlington, MA, USA), D(+)-Maltose (Millipore Sigma, Burlington, MA, USA). Samples were then frozen at −80° C., lyophilized then stored at 25° C. for 3 days. Samples were resuspended in low serum media (OptiMEM, Thermo-Fisher Scientific, Waltham, MA, USA) prior to transfection. To determine the necessity of complexing agents, mRNA was bound to MCMs+/−complexes and lyophilized prior to transfection. For shelf-life studies mRNA were prepared as above and frozen or lyophilized with 25% trehalose, E1, E2, E3 as freezing excipients. Samples in microfuge tubes were placed in a mylar pouch, filled with nitrogen gas, sealed with a heat sealer then placed at −80° C., 4° C., or 25° C. for 3 d-6 months. Samples were moved to −80° C. at each time point prior to transfection. Fresh mRNA complexes were included against the 6-month time points. Transfected media from each time point were collected and assayed immediately with residual media stored at −20° C. Collated media data within each group were normalized to the baseline (d3) data from that group. LNP and LPP samples were bound to regular MCMs or 0.5×F-Cit MCMs (E17 from Table 3) lyophilized and stored at 25° C. for 28 days prior to transfection and subsequent luciferase and metabolic assays.

### Example 16: Binding pH and Zeta Potentials

Fluorescently labeled mRNA were complexed and bound to 0.5×MCMs with dopants F (E12 from Table 3), Cit (E7 from Table 3), and F-Cit (E17 from Table 3), regular MCMs or alone in PBS at varying pHs (2-12) or low serum media. Binding percentages were calculated as described above. Zeta potentials (Zetasizer, Malvern Panalytical, Malvern, UK) were determine at a pH of 4 and 7.4 in PBS for regular MCMs, 0.5×F-Cit MCMs and mRNA complexes alone.

### Example 17: Receptor Inhibitor Studies

To determine the impact of receptor inhibition on MCM and mRNA complex transfection efficacy hMSCs were pre-treated with serial dilutions of drug inhibitors for 2 hours prior to additions of mRNA complexes and MCMs. Media Luciferase activity was assayed 24 hours post transfection and normalized to metabolic activity as assayed by Celltiter Blue assay. Inhibitors used in this study include Nifedipine (Sigma Aldrich, St. Louis, MO, USA; 0-750 μM), a L-type calcium channel blocker, Foscarnet (Sigma Aldrich, St. Louis, MO, USA; 0-1000 μM), a phosphate receptor inhibitor, PF-06761281 a SLC13A5 inhibitor (Sigma Aldrich, St. Louis, MO, USA; Na/Citrate transporter, 0-200 μM) or PSB-603, an adenosine type 2b receptor inhibitor (Tocris Bio, Bristol, UK; 0-100 nM).

### Example 18: Elution Studies

G.Luc mRNA MM LNPs were bound to 0.5×F-Cit MCMs. Initial elution conditions used 0.03-2 mM NaPO₄ in 2 M NaCl (Buffer A, pH 7), 7.5-500 mM NaPO₄ with 150 mM NaCl (Buffer B, pH 6.8), 7.5-400 mM NaPO₄ with 1 mM EDTA (Buffer C, pH 7.0), 7.5-500 mM NaPO₄ (Buffer D, pH 6.8) or 7.5-400 mM KH₂PO₄, 1 mM EDTA (Buffer E). Next changes in EDTA concentration (0-50 mM) and elution time were tested with 30 mM NaPO4 pH 7.0. Following elution minerals were centrifuged at 2000×g for 30 s and then the supernatant was added directly to cells. To test effect of temperature 30 mM NaPO4, 1 mM EDTA was added to 0.5×-FCit MCMs or regular MCMs with bound mRNA complexes and incubated at 60 degrees C. for 30 minutes. Samples were centrifuged as described then diluted 4× in low serum media before addition to hMSCs. Data are luminescence normalized to metabolic activity.

1. A method of preparing a ribonucleic acid (RNA)-based therapeutic composition, comprising:
incubating RNA with a complexing agent to form RNA complexes;
incubating the RNA complexes with a mineral-coated substrate to bind the RNA complexes to the mineral-coated substrate;
suspending the bound RNA complexes in a solution containing a lyoprotectant to provide the RNA-based therapeutic composition; and
lyophilizing the RNA-based therapeutic composition to a dry powder.

2. The method of claim 1, wherein the lyoprotectant is a disaccharide.

3-5. (canceled)

6. The method of claim 1, wherein the mineral-coated substrate includes mineral-coated microparticles (MCMs).

7. The method of claim 6, wherein the method further comprises preparing the mineral-coated microparticles by incubating a modified simulated body fluid (mSBF) with microparticles to form a mineral coating layer on the microparticles.

8. The method of claim 7, wherein the microparticles are beta-tricalcium phosphate (b-TCP) microparticles.

9. The method of claim 1, further comprising storing the dry powder at room temperature prior to addition of a solution, reconstitution and administration of the RNA-based therapeutic composition to a subject in need thereof.

10. The method of claim 1, further comprising reconstituting the dry powder of the RNA-based therapeutic composition in a salt solution and optionally administering the salt solution to a subject in need thereof.

11. The method of claim 1, wherein the mineral-coated substrate is a mineral-coated vial.

12-17. (canceled)

18. The method of claim 1, wherein the RNA is selected from the group consisting of mRNA, microRNA, siRNA, shRNA, iRNA, gRNA, and an aptamer.

19. (canceled)

20. A ribonucleic acid (RNA)-based therapeutic composition, comprising:
a mineral-coated substrate;
RNA complexes bound to the mineral-coated substrate, wherein the RNA complexes include RNA complexed with a complexing agent; and
a lyoprotectant, wherein the RNA-based therapeutic composition is lyophilized to a dry powder.

21. The RNA-based therapeutic composition of claim 20, wherein the lyoprotectant is a disaccharide.

22-26. (canceled)

27. The RNA-based therapeutic composition of claim 20, wherein the mineral-coated substrate includes microparticles coated with a mineral coating layer of calcium, phosphate, and carbonate to provide mineral-coated microparticles (MCMs).

28. (canceled)

29. The RNA-based therapeutic composition of claim 27, wherein the MCMs include beta-tricalcium phosphate microparticles coated with the mineral coating layer.

30. The RNA-based therapeutic composition of claim 20, wherein the mineral-coated substrate is a glass vial having an inner surface coated with a mineral coating layer of calcium, phosphate, and carbonate.

31. (canceled)

32. (canceled)

33. The RNA-based therapeutic composition of claim 20, wherein the RNA is selected from the group consisting of mRNA, microRNA, siRNA, shRNA, iRNA, gRNA, and an aptamer.

34. (canceled)

35. A mineral-coated storage vessel containing a ribonucleic acid (RNA)-based therapeutic composition for administration to a subject in need thereof, comprising:
a glass vial having an abraded inner surface;
a mineral coating layer applied to the abraded inner surface of the glass vial; and
the RNA-based therapeutic composition contained within the glass vial and being in contact with the mineral coating layer, wherein the RNA-based therapeutic composition includes a lyoprotectant and RNA complexed with a complexing agent, and wherein the RNA-based therapeutic composition is lyophilized to a dry powder in the glass vial.

36. The mineral-coated storage vessel of claim 35, wherein the glass vial is a borosilicate glass vial.

37-40. (canceled)

41. The RNA-based therapeutic composition of claim 20, wherein the RNA encodes a severe acute respiratory syndrome coronavirus 2 (SARS COV-2) antigen, a human immunodeficiency virus (HIV) antigen, or a malaria antigen.

42. (canceled)

43. (canceled)

44. The RNA-based therapeutic composition of claim 20, wherein the RNA-based therapeutic composition is a vaccine.

45. The method of claim 1, wherein the complexing agent is selected from a lipid nanoparticle and lipopolyplex.

46. A method of inducing an immune response in a subject comprising administering the RNA-based therapeutic composition of claim 41 to the subject.

47. (canceled)

48. A method of treating a subject in need of expression of a protein comprising administering the RNA-based therapeutic composition of claim 20 to the subject in a therapeutically effective amount.

49. (canceled)

50. (canceled)


SEQUENCE LISTING (8 sequences)

SEQ ID NO: 1 - DNA, 285 bp, synthetic construct
  source 1..285: mol_type = other DNA
ctagagtattaatacgactcactatagggctagcgacatttgcttctgacacaactgtgttcactagcaacctcaaacagacaccagcgctacagtgtacaatgacgatcagcgatcgctcgctttcttgctgtccaatttctattaaaggttcctttgttccctaagtccaactactaaactgggggatattatgaagggccttgagcatctggattctgcctaataaaaaacatttattttcattgcaagtttaaacgaattcgggctcggtacccggggatc

SEQ ID NO: 2 - DNA, 619 bp, synthetic construct
  source 1..619: mol_type = other DNA
ctgtgttcactagcaacctcaaacagacaccatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaagctcgctttcttgctgtccaatttctatta

SEQ ID NO: 3 - DNA, 21 bp, synthetic construct
  source 1..21: mol_type = other DNA
tctctccacaggtgtccactc

SEQ ID NO: 4 - DNA, 23 bp, synthetic construct
  source 1..23: mol_type = other DNA
cacaactgtgttcactagcaacc

SEQ ID NO: 5 - DNA, 21 bp, synthetic construct
  source 1..21: mol_type = other DNA
cctttcgggctttgttagcag

SEQ ID NO: 6 - DNA, 21 bp, synthetic construct
  source 1..21: mol_type = other DNA
ggcagaatccagatgctcaag

SEQ ID NO: 7 - DNA, 30 bp, synthetic construct
  source 1..30: mol_type = other DNA
gctagagtattaatacgactcactataggg

SEQ ID NO: 8 - DNA, 144 bp, synthetic construct
  source 1..144: mol_type = other DNA
ttttttttttttttttttttttttttttttttttttttttttttttttttttttttttttttttttttttttttttttttttttttttttttttttttttttttttttttttttttttttttgcaatgaaaataaatgtttttt